(19) United States
(12) Patent Application Publication
(10) Pub. No.: US 2026/0298918 A1
(43) Pub. Date: Oct. 1, 2026

(54) CORE-SHELL PARTICLES AND METHODS THEREOF

(71) Applicants: Peter C. MAURER, Highland Park, IL (US); Uri ZVI, Chicago, IL (US); Aaron P. ESSER-KAHN, Chicago, IL (US); Michael E FLATTÉ, lowa City, IA (US); Denis R. CANDIDO, lowa City, IA (US); The University of Chicago, Chicago, IL (US)

(72) Inventors: Peter C. Maurer, Highland Park, IL (US); Uri Zvi, Chicago, IL (US); Aaron Esser-Kahn, Chicago, IL (US); Michael E. Flatté, Iowa City, IA (US); Denis R. Candido, Iowa City, IA (US)

(21) Appl. No.: 19/478,590

(22) PCT Filed: Apr. 26, 2024
(86) PCT No.: PCT/US2024/026547

§ 371 Date: Oct. 24, 2025

Related U.S. Application Data
(60) Provisional application No. 63/462,123, filed on Apr. 26, 2023.

(51) Int. Cl. G01N 33/543; B82Y 15/00; B82Y 20/00; B82Y 40/00; C09K 11/02; C09K 11/65

(52) U.S. Cl. CPC G01N 33/54346; C09K 11/02; C09K 11/65; B82Y 15/00; B82Y 20/00; B82Y 40/00

(57) ABSTRACT

The present disclosure relates to core-shell particles, in which a particle can include a core having one or more color centers and a shell disposed around the core. Also provided herein are methods of preparing such particles, devices including one or more core-shell particles, as well as methods for detecting a target using such devices.

### CROSS-REFERENCE TO RELATED APPLICATION

This application claims the benefit of U.S. Provisional Patent Application No. 63/462,123, filed on Apr. 26, 2023, which is incorporated by reference herein in its entirety.

### STATEMENT OF GOVERNMENT INTEREST

This invention was made with government support under DE-AC02-06CH11357 awarded by the U.S. Department of Energy, and OMA1936118, and 2121044 awarded by the National Science Foundation. The government has certain rights in the invention.

### FIELD

The present disclosure relates to core-shell particles, in which a particle can include a core having one or more color centers and a shell disposed around the core. Also provided herein are methods of preparing such particles, devices including one or more core-shell particles, as well as methods for detecting a target using such devices.

### BACKGROUND

Quantum spectroscopy can provide highly precise measurements of ensembles of molecules and individual molecules in diverse areas, including those related to quantum materials, electronic devices, quantum sensors, chemical sensing, and biological sensing.

### SUMMARY

The present document relates to core-shell particles. In some embodiments, the particle includes a diamond core and a shell surrounding the diamond core. Other materials for the core are described herein. The shell can include a metal oxide, a metalloid oxide, a semiconductor material, or an insulator. The present document also relates to systems including such particles (e.g., devices, sensors, and the like), as well as methods of making and using such particles.

Accordingly, in one aspect, the present document relates to a core-shell particle including: a core including one or more color centers; and a shell disposed around the core. In some embodiments, the shell includes a metal oxide, a metalloid oxide, a semiconductor material, or an insulator. In some embodiments, the shell includes silicon oxide, aluminum oxide, titanium oxide, or a combination of any of these.

In some embodiments, the core includes a diamond. In some embodiments, the one or more color centers include a nitrogen vacancy in the diamond. In some embodiments, the core includes silicon carbide, germanosilicate glass, silica, or LiBaF₃.

In some embodiments, the core includes a nanostructure. In some embodiments, the nanostructure includes a nanoparticle.

In some embodiments, an interlayer is disposed on a surface of the core. In some embodiments, the interlayer includes poly(acrylic acid) (PAA), polvvinylpyrrolidone (PVP), polyethylene glycol (PEG), cellulose (e.g., carboxy methyl cellulose), or a combination of any of these.

In one aspect, the present document relates to a device including: one or more core-shell particles (e.g., any described herein); a source configured to irradiate the one or more color centers; and a detector configured to detect one or more output signals emitted from the core-shell particles upon being irradiated.

In one aspect, the present document relates to a method of detecting a target, the method including: providing a sample to an active area of the device (e.g., any described herein); irradiating the device to excite the one or more color centers; and detecting one or more output signals emitted from the core-shell particles upon being irradiated.

In one aspect, the present document relates to a method of detecting a target, the method including: providing a sample to an active area of one or more core-shell particles (e.g., any described herein); irradiating at least one core-shell particle to excite the one or more color centers; and detecting one or more output signals emitted from the at least one core-shell particle upon being irradiated.

In one aspect, the present document relates to a method of preparing a core-shell particle, the method including: providing a core including one or more color centers; and depositing a shell disposed around the core. In some embodiments, the shell includes a metal oxide, a metalloid oxide, a semiconductor material, or an insulator.

In some embodiments, the method further includes (e.g., prior to said providing the core): providing an interlayer on a surface of the core, wherein said depositing the shell includes depositing the shell on a top surface of the interlayer. In some embodiments, the interlayer includes poly(acrylic acid) (PAA), polyvinylpyrrolidone (PVP), polyethylene glycol (PEG), cellulose (e.g., carboxy methyl cellulose), or a combination of any of these. In some embodiments, said depositing the shell includes reacting the top surface of the interlayer with a silanizing agent (e.g., any described herein).

In some embodiments, the core includes a diamond, and wherein the one or more color centers include a nitrogen vacancy in the diamond. In some embodiments, the core includes silicon carbide, germanosilicate glass, silica, or LiBaF₃. In some embodiments, the shell includes silicon oxide, aluminum oxide, titanium oxide, or a combination of any of these. In some embodiments, the core includes a nanostructure (e.g., a nanoparticle). Additional details are provided herein.

As used herein, the term “about” means+/−10% of any recited value. As used herein, this term modifies any recited value, range of values, or endpoints of one or more ranges.

As used herein, the terms “top,” “bottom,” “upper,” “lower,” “above,” and “below” are used to provide a relative relationship between structures. The use of these terms does not indicate or require that a particular structure must be located at a particular location in the apparatus.

Other features and advantages of the present disclosure will be apparent from the following detailed description and the claims.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A-1C provides optical properties of non-limiting bare and core-shell particles. FIG. 1A shows false-colored transmission emission microscopy (TEM) images of bare (top panel) and core-shell (lower panel) particles. Dark gray regions correspond to the diamond core, and light gray regions correspond to the silica shell (for raw TEM images see FIG. 6A). The aggregation of core-shell particles is an artifact of drying the solution on the TEM grid, as discussed herein. FIG. 1B shows correlative light-electron microscopy (CLEM) measurements of fluorescence of core-shell (light gray circles, upper curve in graph) and bare (dark circles, lower curve in graph) particles as a function of core size. Solid lines represent a fit to y=ar³, where r is the core radius and a is a fit parameter. Inset: representative TEM of a bare (i) and a core-shell (ii) particle, labeled as “i” in the lower curve and labeled as “ii” in the upper curve. FIG. 1C shows normalized spectrum (top panel) obtained from an ensemble of core-shell (light gray) and bare (dark gray) particles. Dashed black lines represent deconvolution to NV⁰ (left) and NV⁻ (right) (black lines are extracted from FIG. 1 in Rondin L et al., Phys. Rev. B Condens. Matter, 82:115449 (2010)). Subtraction (lower panel) of the core-shell spectrum from bare spectrum showing a shift from NV⁰ to NV⁻ in core-shell particles. Spectral decomposition reveals that for bare diamond, 24% of the emission originates from NV and for core-shell structures, 29% originates from NV⁻.

FIG. 2A-2D shows electron paramagnetic resonance (EPR) of paramagnetic defects in non-limiting bare and core-shell particles. FIG. 2A shows continuous wave X-band EPR (g=2.0031±0.00005) for bare (dark gray) and core-shell (light gray) diamond nanocrystals with −70 nm core size. FIG. 2B shows spectral deconvolution of the EPR signal into the signal from X (g=2.0032), H1 (g=2.0028), and P1-spins (g=2.0026), normalized to a sample mass of 1 mg. All measurements were performed at room temperature. See methods and other details herein about EPR spectrum modeling. FIG. 2C shows band energy diagram for both Oxygen terminated and SiO₂-coated diamonds with corresponding affinities (χ) and bend alignments. The model shows the corresponding spatial dependence of the Fermi level (Er), bottom of conduction band (E_{c}), top of the valence band (Eᵥ), Nitrogen (P1), and NV center (NV^{-/0}). The energies and occupation of X-spins levels are also indicated. FIG. 2D shows a schematic depiction of a bare (top) and a core-shell (bottom) structured diamond nanocrystal with an NV-qubit (labeled “NV”) in the diamond host. Surface spins are indicated as light gray spheres and arrows.

FIG. 3A-3F shows relaxation and coherence of non-limiting, individually resolvable diamond nanocrystals. FIG. 3A shows single quantum relaxation measurements of bare (dark gray; n=24) and core-shell (light gray; n=23) particles. The inset shows double quantum relaxation times of the same particles (double quantum relaxations were measured at ~6.7 G). FIG. 3C shows maximally obtained T₂ times under Carr-Purcell-Meiboom-Gill (CPMG) dynamical decoupling of randomly selected twelve bare and eight core-shell particles as a function of the number of pulses applied. Note, for some of the investigated bare diamonds T₂ does not increase with N (points with low N), while for all core-shell structured particles, we observed a T₂ for N>1,000 (see, e.g., FIG. 12 and herein for additional details). Solid lines are fits to a power law as described herein. The inset shows a representative coherence time trace data for one bare and core-shell particle with N=1472. FIG. 3B and FIG. 3D show correlation of core size with T₁ (FIG. 3B) and T₂ (FIG. 3D) for four bare and four core-shell particles. FIG. 3E and FIG. 3F show representative CLEM images of bare (FIG. 3E) and core-shell (FIG. 3F) particles for the data shown in FIG. 3B, D. Insets are larger magnifications of individual particles (scale bar=100 nm).

FIG. 4A-4C shows probing spin bath properties using spectral decomposition and stretching factor spectroscopy. FIG. 4A shows spectral decomposition of CPMG data from bare (dark gray) and core-shell (light gray) particles. Gray dotted lines show fits to 1/f^{a} considering DQ data (see FIG. 13A-13E). Black dashed lines show fits for 1/f^{a} plus a single (for bare) or a double (for core-shell) Lorentzian, with the addition of white noise (see FIGS. 13A-13E and details herein for exact fitting details). FIG. 4B shows four representatives bare (left panel) and core-shell (right panel) echo stretching factors. Gray dashed lines are exponential fits for the random walk regime (the cutoff is marked by the black dashed line). Core-shell particles also show ballistic regime fits to n=3. FIG. 4C shows distribution of echo stretching factors for bare (dark gray) and core-shell (light gray). Data points with n≤0.75 (lower dashed box, labeled as “Configurational Averaging”) can be explained by configurational averaging, while data points with n≈1 (upper dashed box, labeled as “Fixed”) correspond to fixed spins' positions (see herein for more details).

FIG. 5A-5B shows growth of non-limiting silica shells on examples of diamond nanocrystals. FIG. 5A shows a two-step shell growth process including a PVP stabilization step and a sol-gel growth step. The enlarged circle to the right illustrates the expected amorphous silica structure containing alcohols and ethyl groups. FIG. 5B shows TEM images of bare (left panel) and core-shell (middle panel) particles. A high-magnification image (right panel) of a diamond nanocrystal encapsulated in silica featuring the diamond crystal lattice lines. Detailed protocol for shell growth are described herein.

FIG. 6A-6D shows characterization of non-limiting bare and core-shell diamond nanocrystals. FIG. 6A shows original TEM images for FIG. 1A. FIG. 6B shows Raman spectra of bare (left panel) and core-shell (right panel) particles featuring diamond peak at 1332 cm⁻¹. FIG. 6C shows XPS spectra of bare (left panel) and core-shell (right panel) particles featuring diamond peak at ~284 eV. FIG. 6D shows XPS spectra of bare (left panel) and core-shell (right panel) particles featuring silica peak at ~101 eV.

FIG. 7A-7F shows TEM grid processing for CLEM and software size analysis. FIG. 7A shows TEM images of bare particles deposited on a UV-ozone treated grid (left panel) and a UV-ozone and PEI treated grid (right panel). FIG. 7B shows a TEM image of bare and core-shell particles on the same grid. Inset shows the binary image produced after processing and thresholding for area calculations. Only fluorescent particles were processed. FIG. 7C shows alignment of TEM and confocal images for CLEM of bare particles used in FIG. 3. FIG. 7D shows an enlarged confocal image containing two bare particles that were used in FIG. 3 (light gray arrow) and one measurement that was disqualified after classification as an aggregate (dark gray arrow). Note that the other two particles used in FIG. 3C are found on a separate confocal scan featured in FIG. 3D. TEM images of individual particles are shown in the panels to the right. FIG. 7E shows alignment of TEM and confocal images for CLEM of core-shell particles used in FIG. 3. FIG. 7F shows enlarged confocal image containing all the core-shell particles that were used in FIG. 3 (light gray arrows), including one measurement that was disqualified after classification as an aggregate (dark gray arrow). TEM images of individual particles are shown in the panels to the right.

FIG. 8A-8D shows EPR of non-limiting bare and core-shell particles. FIG. 8A-8C shows normalization of EPR signals. FIG. 8A shows the mass of bare (left) and core-shell (right) 70 nm and 40 nm particles before (dark shades) and after (light shades) etching with KOH. FIG. 8B-8C shows all normalization factors, F=N_{bare}/N_{CS} for 70 nm (FIG. 8B) and 40 nm (FIG. 8C) particles (see detailed description of the normalization procedure herein). FIG. 8D shows additional EPR data for bare (labeled as “(i)”) and core-shell (labeled as “(ii)”) particles. The upper panel shows raw EPR signals for 70 nm particles before normalization (see FIG. 2A-2B for the normalized signal). The lower two panels show the raw EPR for 40 nm particles (middle panel) and the normalized signal (lower panel). Resonant frequencies for 70 nm particles were 9.392094 GHz and 9.392505 GHz for bare and core-shell particles, respectively. Resonant frequencies for 40 nm particles were 9.395766 GHz and 9.392509 GHz for bare and core-shell particles, respectively.

FIG. 9 shows non-limiting electronic band structures. Provided are the electronic band structure of diamond nanocrystals (left), amorphous silica (middle), and the heterojunction of the core-shell particles (right), leading to the depletion of paramagnetic P1 and X-spins after encapsulation in a silica shell. The modeled bending at the diamond interface is −0.5 eV and 0.225 eV for bare and core-shell particles, respectively. Note that while the illustrated bending in FIG. 2C is exaggerated for better visibility, here it is illustrated true to scale.

FIG. 10A-10D shows non-limiting band bending simulations. FIG. 10A-10B shows simulation results showing the band diagram (FIG. 10A) and the volumetric density of paramagnetic P1 centers (FIG. 10B) as a function of distance from the center, r, in bare diamond nanocrystal with oxygen termination. FIG. 10C-10D shows simulation results showing the band diagram (FIG. 10C) and the volumetric density of paramagnetic P1 centers (FIG. 10D) as a function of r for core-shell nanocrystal. The expected change in P1 is shown in light gray demonstrating a 44% reduction.

FIG. 11A-11F shows non-limiting double electron-electron resonance (DEER) measurements. FIG. 11A shows sequences used for DEER resonance\FID (top), and DEER Rabi (bottom) measurements. The DEER resonance\FID sequence contains four parts. The first two parts are responsible for DEER decay and end with a 180° phase-flipped π/2 pulse to cancel common mode noise (e.g., fluorescence decay due to charge instability). The last two parts of the sequence are responsible for the NV Hahn echo. We used the DEER decay signal, S_{D}=(F1−F2)/(F1+F2), and the Hahn-E_{C} ho decay signal, S_{E}=(F3−F4)/(F3+F4) to extract the DEER FID signal defined as: S_{FID}=S_{D}/S_{E}. To find the DEER resonance, we fixed t=400 ns, while sweeping the microwave frequency. To extract the FID time trace, we set the microwave frequency to the resonance of the paramagnetic defects, while sweeping t. The duration of the 7 pulse is determined by extracting the DEER Rabi frequency. In the Rabi measurement, we fix t, while sweeping the duration of the middle DEER pulse. The signal is defined as S_{Rabi}=(F1−F2)/(F1+F2). The final NV π/2 pulse is phase flipped to cancel common mode noise. FIG. 11B shows detected DEER resonances for bare (dark gray) and core-shell (light gray) particles at ~0.498 GHz. ~0.591 GHz. and ~0.669 GHz, corresponding to expected values for P1 centers and X-spins. FIG. 11C shows DEER Rabi in a core-shell particle confirms coherent driving of paramagnetic defects at 12.8 MHz. FIG. 11D shows spin echo (labeled “E_{C} ho”) and DEER FID (labeled “DEER”) of a bare particle. Inset showing fit results with DEER FID of 0.78 μs. FIG. 11E-11F shows spin echo (light gray) and DEER FID (dark gray) of core-shell particles. Inset showing fit results with DEER FID of 0.99 μs (FIG. 11E) and 0.91 μs (FIG. 11F). The particle in FIG. 11E presents nuclear spin oscillations likely resulting in shorter spin echo time. All measurements were performed at ~205 G.

FIG. 12A-12F shows non-limiting T₂ measurements. FIG. 12A shows a full version of FIG. 3B featuring all CPMG measurements for bare (dark gray) and core-shell (light gray) particles. Closed markers represent the maximum T₂ measured for a given particle. Solid lines are fits to T₂(N)=T_{2,echo}N^{k}, considering all measurements for a given group. Since all core-shell particles showed measurable improvement for the highest number of pulses measured (N>1000), the fitted parameter k=0.53 is a representation of the average core-shell particle. Only two bare particles continued to show improvement for N>1000, and most saturated at a much smaller number of pulses. Therefore, the fitted parameter k=0.47 represents a higher bound for bare particles that showed a large distribution of k=0 to 0.47. The sequence used for CPMG measurements is shown in the inset (initialization pulse was ignored). The T₂ decay signal was recorded as S_{CPMG}=(F1−F2)/(F1+F2) and fitted to the time-dependent coherence C(t)=ae^{-χ(t)}, where we defined

$$χ ( t ) = {( \frac{t}{{T}_{2}} )}^{n} .$$

FIG. 12B shows Monte Carlo simulations assessing the effect of dephasing during the π-pulses. We set N=10, τ_{C}=1 ns, and t, =10 ns to represent the scenarios in our CPMG measurements, where τ_{C}≤t_{π} and Nt_{π} is on the same order of magnitude as T₂ (see discussion for more simulation details). The exact numerical (dashed curves) and the C(t)≈exp[−S(ω₀)t]approximated (solid curves) coherence signals were calculated assuming no dephasing during the pulse duration (labeled as “(iii)” and “(iv)”) and for an infinitesimally short pulse, t_{π}=0 ns (labeled as “(i)” and “(ii)”). FIG. 12C shows T₂ echo signals for the eight measured core-shell particles, with an average T₂^{E}_{C} ho=1.42±0.20 μs. FIG. 12D shows T₂ CPMG signals for the eight measured core-shell particles. FIG. 12E shows T₂ echo signals for eleven measured bare particles (ND5 E_{C} ho T₂ wasn't recorded due to an error), with an average T₂^{E}_{C} ho=1.05±0.41 μs. FIG. 12F shows T₂ CPMG signals for the twelve measured bare particles.

FIG. 13A-13E shows noise spectrum fitting. All points obtained with the noise spectral density extraction procedure for bare (FIG. 13A) and core-shell (FIG. 13B) particles before binning. FIG. 13C shows that the data can be grouped to 14 logarithmic bins. A single Lorentzian fit to equation (1) did not fit well for both groups (bare: Δ=1.4e7, τ c=3 ns, rsquare=0.06; core-shell: Δ=6. 1e6, τ c=17 ns, rsquare=0.63). FIG. 13D shows a double Lorentzian fit described well only the core-shell group (bare: Δ₁=3.4e6, τ_{C1}=44 ns. Δ₂=2.2e7. τ_{C2}=0.9 ns, rsquare=0.53; core-shell: Δ₁=3.8e6, τ_{C1}=46 ns, Δ₂=1.1e7, τ_{C2}=1.7 ns, rsquare=0.96). Light gray dashed lines represent the bare particles' fit for the Lorentzians with short τ_{C1} (light gray dashed line extending to the right past 100 MHz in FIG. 13D) and long τ_{C1} (light gray dashed line extending to the right until about 5 MHz in FIG. 13D). It is clear that the short τ_{C} Lorentzian is dominant, and that a good fit can only be obtained by adding a Lorentzian with much longer τ_{C}, in the regime dominated by electric noise. FIG. 13E shows that a fit for both groups was obtained by considering electric field noise and fitting 1/f-like noise using the DQ relaxation data and equation (2). (bare: Δ =2.4e7, τ_{C}≤1 ns, a=1.7, rsquare=0.75; core-shell: Δ_{C,1}=2.9e6, τ_{C1}=40 ns, Δ_{C,2}=1.3e7, τ_{C,2}≤1 ns, a=1.6, rsquare=0.95). The fitted a is within the expected range, 1≤α≤2, arising from one or more Lorentzian noise baths, and agrees well with previous reports. All dashed black lines are the fits to the total data. Only CPMG data for pulse number N>64 was considered to uphold the approximation of the filtering function as a delta function, as described herein.

FIG. 14 shows non-limiting echo stretch factors. All bare (left panel) and core-shell (right panel) echo stretching factors were extracted as described in FIG. 4C. Gray dashed lines are exponential fits for the random walk regime (the cutoff is marked by the black dashed line). For bare particles, the cutoff is too short to plot (30 ns). Core-shell particles also show ballistic-regime fits to n=3 to guide the eye (see methods and details herein for more details about cutoff choice and plotting procedure).

### DETAILED DESCRIPTION

The present disclosure relates to core-shell particles characterized by, e.g., increased coherence of nitrogen-vacancy centers, reduced heterogeneity, improved qubit coherence, reduced surface states, and/or improved sensing sensitivity. In turn, such particles can be employed to provide devices and systems for quantum sensing. Non-limiting devices and systems can include, e.g., qubit systems, nanosensors (e.g., in intact cells or living organisms), photonic systems, atomic force microscopy systems, and the like.

In some embodiments, the shell comprises a metal oxide or metalloid oxide capping layer. In some embodiments, the shell (e.g., a capping layer) provides improved spin coherence through surface modification. In particular embodiments, the particle comprises a qubit (e.g., a nanoscale qubit). In other embodiments, improvement of spin coherence using a shell is provided in a qubit system (e.g., a nanoscale cubit system).

Methods of making and using such particles are also described herein. Accordingly, the present document encompasses methods of making such particles, e.g., by providing metal-oxide shell growth on a core (e.g., a diamond core). The present document also encompasses methods of using such particles, e.g., by detecting one or more signals from core-shell particles (e.g., any described herein), wherein the one or more signals are indicative of the presence of a target.

In some embodiments, the methods herein can be used to engineer the spin coherence of solid-state qubits through modification of their surface. Such surface modification can include providing a shell having a metal oxide or a metalloid oxide. Without wising to be limited by mechanism, the surface modification can lead to saturation of surface states through band bending (and subsequent state occupation or de-occupation), direct conjugation to dangling bonds, and/or displacement of paramagnetic species. In turn, this can result in reduction of magnetic noise and improved qubit coherence times, which can lead to higher sensing sensitivity and reduced signal integration time. Additionally, the modification may lead to decreased spin mobility at the surface.

This approach can be employed for a core including any useful material. In some embodiments, the material includes diamond. In other embodiments, the material can include one or more color centers (e.g., a nitrogen-vacancy). Color centers generally include defects within transparent, crystalline insulators or large band-gap semiconductors, such as diamond, silicon carbide, germanosilicate glass, silica, or LiBaF₃. Such defects can include point defects; substitution defects in which an atom within the substrate is replaced with another atom; and vacancy defects in which an atom is missing within the crystalline lattice, as well as combinations thereof (e.g., nitrogen-vacancy (NV) centers in diamond having a nitrogen substitution in proximity to a vacancy, germanium-related detects in germanosilicate glass, silicon vacancies silicon carbide, and the like). Such color centers be probed via nanoscale nuclear magnetic resonance spectroscopy (NMR), optical detection of magnetic resonance (ODMR), or other techniques. The core can be in any useful form (e.g., a particle, such as a nanoparticle; a quantum dot; a crystal, such as a nanocrystal; as well as other nanostructures or microstructures). Other non-limiting examples of materials are described in Int. Pub. No. WO 2023/288108, which is incorporated herein by reference in its entirety.

In some instances, the core comprises a diamond having one or more color centers at a depth of less than about 100 nm from the top surface of the substrate. Such color centers can be one or more NV centers. In other instances, at least one color center is at a depth of about 0.1 nm to about 100 nm from the top surface of the substrate (e.g., a depth of about 0.1 to 90 nm, 0.1 nm to 70 nm, 0.1 to 50 nm, 0.1 to 40 nm, 0.1 to 30 nm, 0.1 to 20 nm, 0.1 to 10 nm, 0.1 to 5 nm, 0.2 to 100 nm, 0.2 to 90 nm, 0.2 to 70 nm, 0.2 to 50 nm, 0.2 to 40 nm, 0.2 to 30 nm, 0.2 to 20 nm, 0.2 to 10 nm, 0.2 to 5 nm, 0.5 to 100 nm, 0.5 to 90 nm, 0.5 to 70 nm, 0.5 to 50 nm, 0.5 to 40 nm, 0.5 to 30 nm, 0.5 to 20 nm, 0.5 to 10 nm, 0.5 to 5 nm, 1 to 100 nm, 1 to 90 nm, 1 to 70 nm, 1 to 50 nm, 1 to 40 nm, 1 to 30 nm, 1 to 20 nm, 1 to 10 nm, 1 to 5 nm, 1.5 to 100 nm, 1.5 to 90 nm, 1.5 to 70 nm, 1.5 to 50 nm, 1.5 to 40 nm, 1.5 to 30 nm, 1.5 to 20 nm, 1.5 to 10 nm, 1.5 to 5 nm, 2 to 100 nm, 2 to 90 nm, 2 to 70 nm, 2 to 50 nm, 2 to 40 nm, 2 to 30 nm, 2 to 20 nm, 2 to 10 nm, 2 to 5 nm, 5 to 100 nm, 5 to 90 nm, 5 to 70 nm, 5 to 50 nm, 5 to 40 nm, 5 to 30 nm, 5 to 20 nm, or 5 to 10 nm, from the top surface of the substrate). In particular embodiments, the core comprise a diamond having an oxygen-tenninated surface and stable NV centers in proximity to this surface.

The shell can include any useful material. Non-limiting examples of such materials include a metal oxide, a metalloid oxide, a semiconductor material, or an insulator. In some embodiments, the shell comprises silicon oxide (e.g., silicon dioxide), aluminum oxide, titanium oxide, and the like. In some embodiments, the shell is provided by depositing a silicon precursor. Non-limiting silicon precursors include a silane, such as silicon alkoxides, including tetramethyl orthosilicate (TMOS), tetraethyl orthosilicate (TEOS), tetrapropyl orthosilicate (TPOS), and the like.

In some embodiments, the core-shell particle can include the use of a functionalized core. The functionalized core can include a core material (e.g., any described herein) surrounded by an interlayer. The interlayer may be located between the core and the shell, between the core and an adhesion layer, and/or between the adhesion layer and the shell. Any number of interlayers can be located between the core and the shell, between the core and the adhesion layer, and/or between the adhesion layer and the shell. The interlayer can include, for example and without limitation, poly(acrvlic acid) (PAA), polyvinylpyrrolidone (PVP), polyethylene glycol (PEG), cellulose (e.g., carboxy methyl cellulose), and the like.

To provide an interlayer, deposition can include the use of a reagent to react with a top surface of a substrate (e.g., a core, an adhesion layer, and/or a shell). Such deposition can include conditions or reagents that provide a surface (e.g., a reactive surface) upon which further layers can be attached to the interlayer. Optionally, the surface can include one or more reactive moieties, which are in turn provided by the reagent. In one embodiment, the reagent is a monomer or other polymer precursor, which can be polymerized to form a polymeric layer (e.g., a polymeric interlayer). In one embodiment, the reagent is a silanizing agent (e.g., an organosilane, a silane, etc.) having at least one reactive moiety to react with an underlying surface (e.g., of a substrate, such as a core, adhesion layer, another interlayer, and/or a shell) and another reactive moiety to react with an overlaying surface (e.g., of an adhesion layer, another interlayer, a shell, and/or a functionalized layer). Non-limiting examples of silanizing agents include. e.g., silazane (e.g., hexamethyldisilazane (HMDS)), haloalkylsilane (e.g., methyltrichlorosilane, trichlorocyclohexylsilane, dichlorodimethylsilane, dichloroethylsilane, bromotrimethylsilane, or chlorotrimethylsilane), haloarylsilane (e.g., fluorotriphenylsilane), trialkylsilylsilane (e.g., chlorotris(trimethylsilyl)silane), and silanol (e.g., 2-(trimethylsilyl)ethanol).

In some embodiments, the core-shell particle can include the use of a treated core. The treated core can include a core material (e.g., any described herein) that has been irradiated and/or annealed.

In some embodiments, the core-shell particle can include the use of an adhesion layer disposed on the top surface of the core, the interlayer, and/or the shell. The adhesion layer may be located between the core and the shell, between the core and an interlayer, and/or between the interlayer and the shell. Any number of adhesion layers can be located between the core and the shell, between the core and the interlayer, and/or between the interlayer and the shell. The adhesion layer can include any useful material, such as, e.g., an oxide (e.g., a silanizable oxide, an aluminum oxide, a silicon oxide, a titanium oxide, a patterned oxide, or the like). The adhesion layer can be disposed on a portion of the top surface of the core, the interlayer, and/or the shell. In some embodiments, the adhesion layer can be disposed on a majority of the top surface of the core. In some non-limiting instances, the adhesion layer can be disposed on substantially the entirety of the top surface of the core, the interlayer, and/or the shell. Optionally, the adhesion layer itself can be patterned, thereby providing a patterned adhesion layer having exposed portions (thereby providing exposed regions of the underlying substrate, which can be the core, the interlayer, and/or the shell) and non-exposed portions (thereby providing covered regions overlying the substrate, which can be the core, the interlayer, and/or the shell, in which the covered regions are composed of the material for the adhesion layer).

The adhesion layer can be deposited in any useful manner, including chemical vapor deposition (CVD), atomic layer deposition (ALD, e.g., thermal ALD and plasma-enhanced ALD), physical vapor deposition (PVD), or molecular layer deposition (MLD), plasma-enhanced fonns thereof, sputter deposition, e-beam deposition including e-beam co-evaporation, etc., or a combination thereof, such as ALD with a CVD component, such as a discontinuous, ALD-like process in which metal- or metalloid-containing precursors and oxygen-containing reactants are separated in either time or space. To provide an oxide layer, deposition can include the use of a metal- or metalloid-containing precursor with an oxygen-containing reactant. For example, the metal- or metalloid-containing precursor and oxygen-containing reactant can be introduced at separate times, representing an ALD cycle. The precursor can react on the surface, forming up to a monolayer of material at a time for each cycle. An oxygen-containing reactant may be pulsed between the precursor pulses resulting in ALD or ALD-like growth of the oxide layer. In other cases, both the precursor and the oxygen-containing reactant may be flowed at the same time. The adhesion layer may be patterned. In one instance, such patterning can include etching of the adhesion layer; immobilizing functional moieties (e.g., reactive or unreactive moieties) on a top surface of the adhesion layer; and the like.

Any portion of the top surface of a substrate may be covered by any layer described herein (e.g., a core, an interlayer, an adhesion layer, a functionalized layer, and/or a shell). In some embodiments, only a portion of the top surface of the substrate is covered by a layer (e.g., any layer described herein). In other embodiments, a substantial portion or all of the top surface of the substrate is covered by a layer (e.g., any layer described herein).

Any particle herein can be provided within a device. The device can include one or more additional components to allow for detection of a target. For instance, the device can include a source (e.g., an optical source and/or a microwave source) configured to irradiate the substrate and/or the one or more color centers; and a detector (e.g., an optical detector) configured to detect one or more output signals emitted from the substrate upon being irradiated. Other components include filters, lenses, phase shifters, and the like.

Methods employing such particles are also described herein. For example, methods herein can be used for detecting one or more targets. In some non-limiting embodiments, detection of the target is conducted in a label-free manner. For example, the target of interest can be free of labels, and selectivity for the target can be provided by the capture agent(s). Non-limiting targets include a biomolecule (including a tagged biomolecule), a nucleic acid (e.g., e.g., oligonucleotides, polynucleotides, nucleotides, nucleosides, molecules of DNA, or molecules of RNA, including a chromosome, a plasmid, a viral genome, a primer, or a gene), a peptide, a protein, a receptor, a ligand, a toxin, a cell, a tissue, a bacterium, a virus, a pathogen, a microorganism, an allergen, as well as components thereof (e.g., a modification, a polymorphism, a structural configuration such as folding or misfolding configuration of a nucleic acid, a peptide, or a protein). In other embodiments, the target is a chemical, a small molecule, a pharmaceutical, and the like.

The target can be present in any useful sample. Non-limiting samples can include a microorganism, a virus, a bacterium, a fungus, a parasite, a helminth, a protozoon, a cell, tissue, a fluid, a swab, a biological sample (e.g., blood, serum, plasma, saliva, etc.), a plant, an environmental sample (e.g., air, soil, and/or water), etc.

Optionally, the device can include a capture agent that binds to the biomolecule, the tagged biomolecule, or a portion thereof. One non-limiting method includes providing a sample to a particle (e.g., any described herein); irradiating the device to excite the one or more color centers (e.g., by use of a source); and detecting one or more output signals emitted from the substrate upon being irradiated (e.g., by use of a detector). In some embodiments, the sample provided to the active area comprises a biomolecule and/or a physiological buffer.

An active area can include a portion of a device including one or more core-shell particles (e.g., any described herein) or a portion of a core-shell particle. In one non-limiting instance, the active area includes one or more active sites, and the inactive area lacks active sites. In another non-limiting instance, the active area includes the functionalized layer, and the inactive area lacks the functionalized layer. In some embodiments, a density of active sites and inactive sites in the active area can be controlled. In some embodiments, an active area and/or active sites can include one or more capture agents, which in turn allow for binding to targets. In this way, the active area can be used for sensing and detection (e.g., of one or more targets). The active area and inactive areas can be patterned, for example, in a configuration suitable for multiplexing or high-throughput applications.

The devices and methods herein can be used with any other useful component. Non-limiting components include a source (e.g., configured to provide radiation to the substrate, including excitation light, microwave radiation, and the like), a detector (e.g., configured to detect an optical emission from the substrate, an emitted radiation, and the like, as well as frequency and/or wavelength measurements), a fluidic device (e.g., configured to provide a sample or a target to a capture agent, such as a well, a microfluidic device, and the like), a sample holder, a manifold, and the like.

Non-limiting sources include a pulsed source (e.g., a pulsed optical source or a pulsed microwave source), a microwave/radiofrequency electromagnetic field source, an optical source (e.g., a laser or a light emitting diode), a microwave source (e.g., a tuned microwave source), and the like. Non-limiting detectors include a photodetector, an electronic detector, or an optoelectronic detector. The fluidic device can include any fluidic structure configured to provide fluidic communication to a surface of the substrate. Such fluidic structures can include a channel, a well, a chamber, an access port, a reservoir, and the like.

Methods of making the device are also described herein. Such a method can include, without limitation, providing a substrate (e.g., a core comprising one or more color centers); and depositing a shell on a top surface of a substrate (e.g., as described herein), wherein the substrate comprises one or more color centers in proximity to the top surface. The shell can be provided directly or indirectly on a top surface of the core. In some embodiments, the shell is deposited on a top surface of the core, the interlayer, the adhesion layer, or combinations thereof. In some embodiments, a functionalized layer is deposited on a top surface of the shell.

The method can include one or more further operations, such as, e.g., providing an interlayer on a surface of the core, wherein said depositing the shell comprises depositing the shell on a top surface of the interlayer. In some embodiments, the method includes providing an interlayer on a surface of a substrate (e.g., a core, another interlayer, and/or an adhesion layer). One or more further layers can be deposited on the interlayer. In some embodiments, the method includes reacting a top surface of the substrate (e.g., a core, an adhesion layer, and/or a shell) with a reagent (e.g., a monomer or other polymer precursor) to provide an interlayer (e.g., any described herein).

In some embodiments, the method includes providing an adhesion layer on a surface of a substrate (e.g., a core, an interlayer, and/or another adhesion layer). One or more further layers can be deposited on the adhesion layer. In some embodiments, the method includes reacting a top surface of the substrate (e.g., a core, an interlayer, and/or a shell) with a reagent (e.g., a silanizing agent) to provide an adhesion layer, wherein a top surface of the adhesion layer comprises a reactive moiety.

In some embodiments, the method includes attaching a functionalized layer to a substrate (e.g., a core, an adhesion layer, an interlayer, and/or a shell), wherein the functionalized layer comprises one or more capture agents configured to capture a target. In some embodiments, attaching the functionalized layer includes providing a poly(ethylene glycol) group optionally comprising the one or more capture agents. In other embodiments, attaching the functionalized layer includes providing a poly(ethylene glycol) group having a further reactive moiety, and then providing one or more capture reactants to react with the further reactive moiety.

Deposition of a material, as well as patterning or treating a layer of the material, can include any useful process. Exemplary processes include polymerization; coating, such as spin coating, thin film coating, etc.; casting; laminating; epitaxial growth; polishing, such as chemical-mechanical polishing (CMP); chemical vapor diffusion (CVD), such as metal-organic CVD (MOCVD), metal-organic vapor phase epitaxy (MOVPE), plasma-enhanced chemical vapor deposition (PECVD), and molecular beam epitaxy (MBE); milling (e.g., ion milling or focused ion beam milling); rapid prototyping; microfabrication (e.g., by casting, injection molding, compression molding, embossing, ablation, thin-film deposition, and/or Computer Numerically Controlled (CNC) micromachining); photolithography; atomic layer deposition (ALD); and etching techniques (e.g., wet chemical etching, reactive ion etching (RIE), deep RIE, sputter etching, inductively coupled plasma deep silicon etching, buffered oxide etching (BOE), laser ablation, or air abrasion techniques). In one instance, the adhesion layer can be constructed of any oxide material deposited via physical or electrochemical deposition, which can be optionally patterned or implanted.

A surface of a material can be further reacted or functionalized in any useful manner. In one instance, the surface can be reacted with an agent to provide a reactive moiety. Non-limiting agents can include silanizing agents, PEGvlating agents, and the like. Such agents can provide a linker (e.g., to which additional moieties or capture agents can optionally be added), a reactive moiety, a capture agent, and the like. Alternatively, the surface can be reacted with an agent to provide a biocompatible or cytocompatible moiety. Non-limiting examples of such agents include a biocompatible polymer, a biopolymer such as chitosan, a cationic polymer, an antifouling polymer, and the like. For instance and without limitation, biocompatible polymers can include poly(ethylene glycol); polv(lactic acid) (PLA) including poly(DL-lactic acid) (DL-PLA), poly(L-lactic acid) (L-PLA), and poly(D-lactic acid) (D-PLA); poly(glycolic acid) (PGA); poly(lactic-co-glycolic acid) (PLGA) including poly(DL-lactic-co-glycolic acid) (DL-PLGA); a poly(ester), such as polyhydroxybutyrate, polyhydroxyvalerate, or copolymers thereof; polv(vinyl alcohol); poly(dioxanone); poly(caprolactone); poly(orthoester); poly(anhydride); poly(phosphazine); poly(propylene carbonate); poly(propylene succinate); poly(urethane); as well as copolymers thereof). In other embodiments, the agent can be poly(imide), benzocyclobutene, glass, a fluoropolymer (e.g., a fluoroacrylate or polytetrafluoroethylene), a photoresist, and the like.

For example, in certain embodiments, the method includes atomic layer deposition of Al₂O₃ onto a pristine, oxygen-terminated diamond surface, followed by silanization (to provide a first linker attached to the diamond surface and to present a terminal reactive group), and then PEGylation (e.g., with a poly(ethylene glycol) group comprising the one or more capture agents, or with a poly(ethylene glycol) group having a further reactive moiety, in which the PEG group can react with the terminal reactive group provided by way of silanization). In another example, in certain embodiments, the method includes atomic layer deposition of TiO₂ onto a pristine, oxygen-terminated diamond surface, followed by silanization and PEGylation (e.g., with a poly(ethylene glycol) group comprising the one or more capture agents, or with a poly(ethylene glycol) group having a further reactive moiety).

The functionalized layer can include one or more capture agents. One or more capture agents can be selected from the group of a nucleic acid (e.g., a nucleotide, a single stranded DNA, a single stranded RNA, and an oligonucleotide, including modified forms of any of these, as well as hairpin forms or double-stranded forms of these; also including a polythymine), a peptide (e.g., a polypeptide, including modified forms thereof, such as glycosylated polypeptides or multimeric polypeptides), a protein (e.g., avidin, streptavidin, neutravidin, an enzyme, a receptor, and the like), a cofactor (e.g., biotin or a metal ion such as Ni²⁺), a receptor, an enzyme, an antibody (e.g., including monoclonal or polyclonal forms thereof, an affibody, or fragments or recombinant forms of any of these), an affibody, a lectin, and a click chemistry moiety (e.g., an azido group, an alkynyl group, a dienophile group, or a diene group).

A peptide can include a polyhistidine (e.g., including 6-9 histidine residues) or a polyglycine (e.g., including 4-6 glycine residues). Other peptides may be employed, such as those that can be used as an affinity tag. Non-limiting affinity tags include a polyhistidine tag, a polyarginine tag (e.g., including 4-6 arginine residues), glutathione-S-transferase (GST), a FLAG tag (e.g., including a combination of aspartic acid, tyrosine, and lysine), a streptavidin-binding protein, a streptavidin binding tag, a modified streptavidin-binding tag, a calmodulin binding peptide (CBP) tag, a chitin-binding domain (CBD) tag, maltose-binding protein (MBP) tag, as well as combinations thereof or modified forms thereof.

A click chemistry moiety can include those from a click-chemistry reaction pair selected from the group consisting of a Huisgen 1,3-dipolar cycloaddition reaction between an alkynyl group and an azido group to form a triazole-containing linker; a Staudinger reaction between an azido group and a phosphine or phosphite to form a iminophosphorane-containing linker; a Diels-Alder reaction between a diene having a 4π electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 2π electron system (e.g., an optionally substituted alkenyl group or an optionally substituted alkynyl group); a ring opening reaction with a nucleophile and a strained heterocyclyl electrophile; a splint ligation reaction with a phosphorothioate group and an iodo group; a reductive amination reaction with an aldehyde group and an amino group; and a Michael addition reaction between a thiol group and a maleimide group, as well as variants of any of these.

In certain embodiments, the click chemistry moieties are conducted in a copper-free condition. In one example, a click chemistry moiety can include those from a click-chemistry reaction pair selected from the group consisting of a Huisgen 1,3-dipolar cycloaddition reaction between a cyclic alkynyl (or cycloalkynyl) group (e.g., a cyclooctvnyl group, including optionally substituted forms thereof, such as halo substituted forms) and an azido group to form a triazole-containing linker. In certain embodiments, the cyclic alkynyl group can further include one or more heteroatoms (e.g., nitrogen, oxygen, sulfur, and the like).

The one or more capture agents can include moieties from one or more bioconjugate pairs, for example, one or more moieties configured to form a covalent link between a capture agent and a biomolecule. Bioconjugate pairs can include, for example, biotin and a biotin-binding biomolecule (e.g., avidin, streptavidin), maleimide and a thiol-containing biomolecule (e.g., a cysteine-containing biomolecule), a metal ion (e.g., Ni²⁺) and a histidine-containing biomolecule (e.g., a polyhistidine-tagged biomolecule), a polyglycine and a biomolecule containing a sortase signal, and click chemistry pairs such as an azido group and an alkynyl-containing biomolecule (e.g., a DBCO-tagged biomolecule).

The capture agents can be distributed spatially homogeneously as a monolayer throughout the functionalized layer. In some embodiments, the average number of capture agents present per μm² of the functionalized layer is less than 10, or less than 7.5, or less than 5. For example, the functionalized layer can include an average of about 0.01 to about 10, about 0.01 to about 7.5, about 0.01 to about 5, about 0.1 to about 10, about 0.1 to about 7.5, about 0.1 to about 5, about 0.1 to about 2.5, or about 0.1 to about 1 capture agents per μm². In other examples, the functionalized layer can include up to about 1,000, up to about 10,000, or up to about 100.000 capture agents per μm². In particular examples, the functionalized layer can include up to about 1 capture agent per nm².

### EXAMPLES

### Example 1: Engineering Spin Coherence in Core-Shell Diamond Nanocrystals

Diamond nanocrystals can harbor spin qubit sensors capable of probing the physical properties of biological systems with nanoscale spatial resolution.¹ These diamond nanosensors can readily be delivered into intact cells² and even living organisms.³ However, applications beyond current proof-of-principle experiments would benefit from a substantial increase in sensitivity, which is generally limited by surface-noise-induced spin dephasing and relaxation.⁴ As described herein, we significantly reduce magnetic surface noise by engineering core-shell structures, which in combination with dynamical decoupling result in qubit coherence times (T2) ranging from 52 μs to 87 s—an improvement over the 1.1 μs to 35 μs seen in bare particles. This improvement in spin coherence, combined with an overall increase in particle fluorescence, corresponds to a two-order-of-magnitude reduction in integration time. Probing qubit dynamics at a single particle level, furthermore, reveals that the noise characteristics can fundamentally change from a bath with spins that rearrange their spatial configuration during the course of an experiment to a more dilute static bath. The observed results shed light on the underlying mechanisms governing spin dephasing in diamond nanocrystals and offer an effective noise mitigation strategy based on engineered core-shell structures.

Nitrogen vacancy (NV) centers in diamond nanocrystals have emerged as a powerful platform for sensing magnetic fields,^{5,6} electric fields,⁷ and temperature^{8,9} in living systems. First applications of diamond-based sensing are emerging in neuroscience,¹⁰ developmental biology,⁹ cellular physiology,^{11,12} and medical diagnostics.¹³ However, many potential applications relying on diamond nanocrystal-based sensing remain limited by relatively short NV T₂-times (e.g., which are typically 100× shorter than what is observed in high-purity bulk crystals¹⁴). Specifically for particles with a size below 100 nm, charge and spin noise associated with the crystal's surface can become a dominant factor.⁴ Various approaches to extending the spin coherence in diamond micro- and nanocrystals have been pursued. Notably, these approaches include mechanical milling^{15,16} or lithographically¹⁷ etching high-purity bulk diamond to microscale particles, which under dynamical decoupling, results in bulk-like T₂-times. However, the microscale size (hundreds of nanometers^{15,16} in the case of milling and pillar length of a few micrometers¹⁷ in the case of lithographic etching) of these particles can severely limit biological applications. Moreover, the top-down fabrication required in lithography results in a low yield, limited to tens of micrograms, which makes processing large quantities prohibitively expensive. Another approach towards the mitigation of surface noise relies on controlling the diamond surface termination, which on highly ordered bulk diamond surfaces, has led to a significant increase in spin coherence.¹⁸ However, in diamond nanocrystals a similar effect on coherence has yet to be observed.

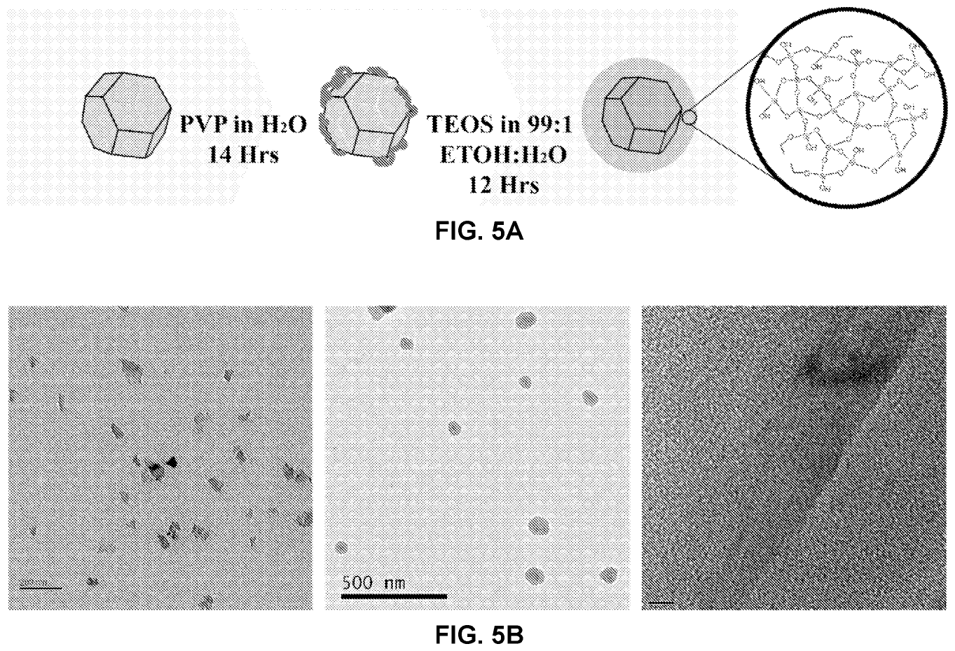

In nanotechnology, engineered core-shell structures could mitigate adverse surface effects on luminescence. For example, encapsulation in a protective shell can reduce surface-induced photoblinking in quantum dots¹⁹ and non-radiative relaxation in lanthanide-doped upconverting nanoparticles.^{20,21} Without wishing to be limited by mechanism or theory, a coating strategy could lead to enhanced qubit coherence by saturating dangling bonds and eliminating paramagnetic defects or charge traps located near the particle's surface.²¹ However, extending qubit coherence in core-shell structured particles has so far remained elusive.^{4,22} As described herein, we explore core-shell structures to efficiently passivate the diamond surface. This leads to a T₂ extension in diamond nanocrystals that rivals those of near-surface NVs in high-purity bulk crystals.¹⁸ As a substrate, we use electron irradiated and thermally annealed (e.g., at 850° C.) carboxylated diamond nanocrystals with a reported average diameter of 44 nm and 10-12 NV centers per particle.²³ The diamond nanocrystals were coated with 18±2 nm silica shells using an adapted Stober process with tetraethyl orthosilicate (TEOS) to obtain core-shell structured particles,²⁴ resulting in milligrams of diamond core-shell particles (see, e.g., FIG. 5 and details herein).

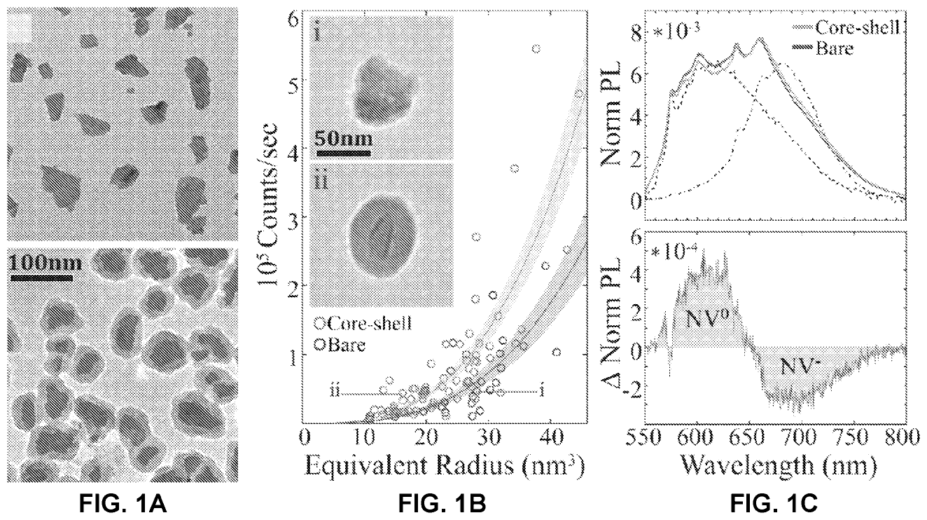

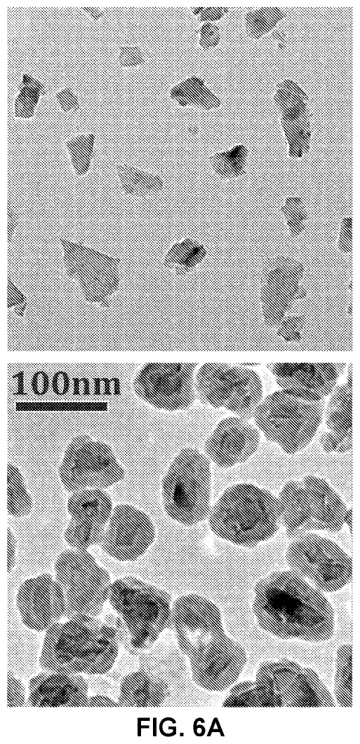

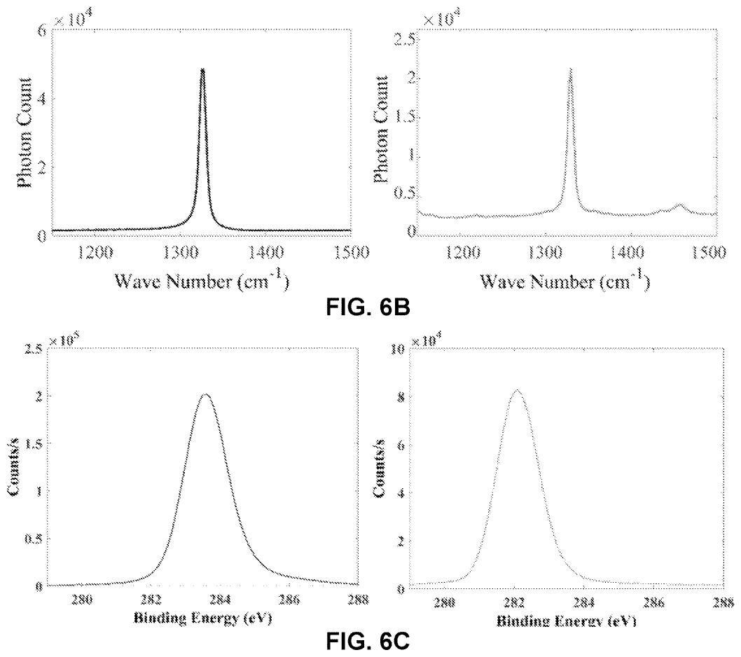

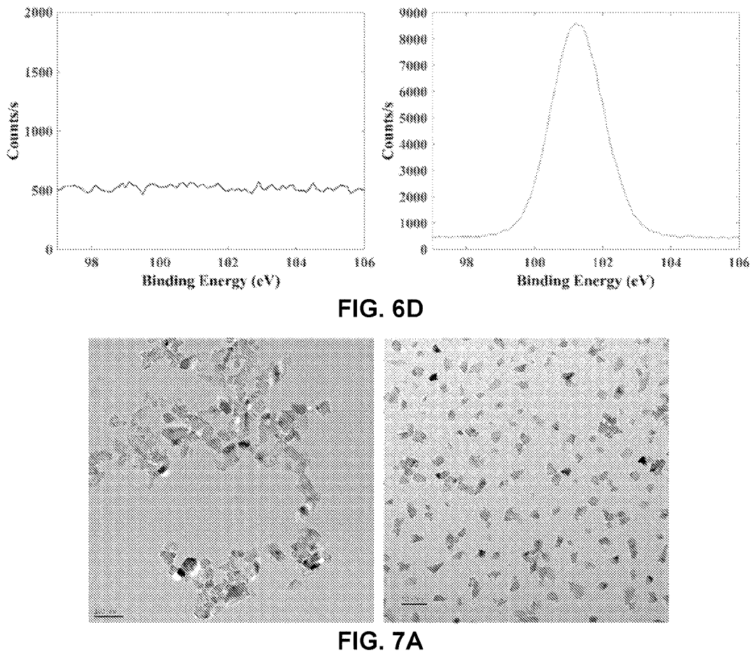

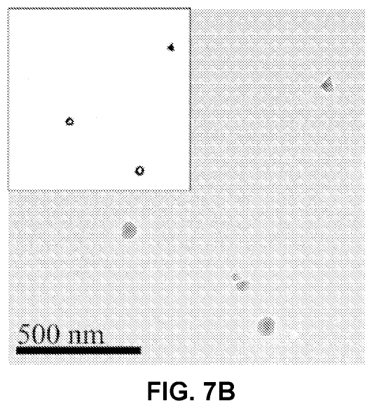

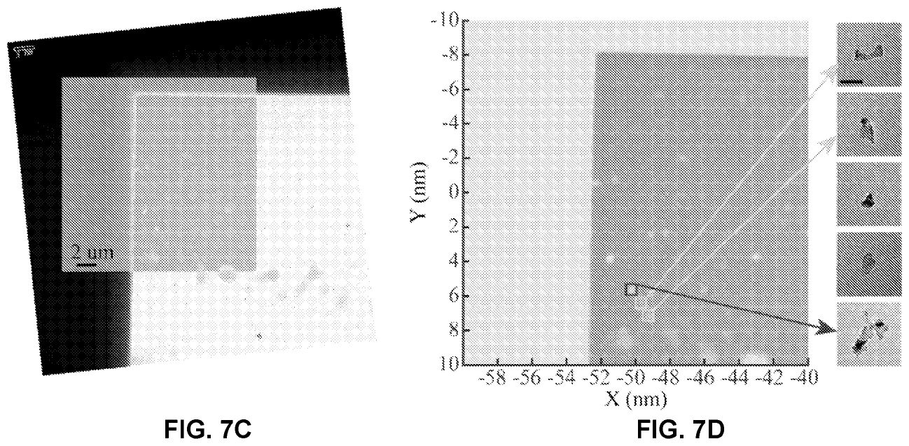

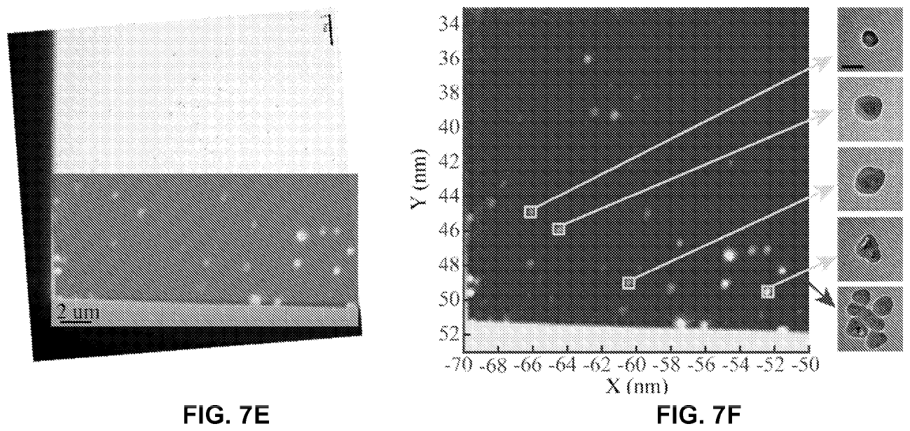

Transmission electron microscopy (TEM) reveals dense silica shells with homogeneous surface coating for these core-shell structures (FIG. 1A and FIG. 6). Correlative light-electron microscopy (CLEM) imaging reveals that core-shell structures result in a 1.85-fold increased luminescence for a given diamond core size (see, e.g., FIG. 1B and FIG. 7 and other details herein). Fluorescence spectroscopy confirms that our core-shell structures stabilize the desired negatively charged NV center (FIG. 1C). Deconvolution of the fluorescence spectra of NV⁰ and NV⁻ suggests a 20% increase of NV⁻. Without wishing to be limited by mechanism or theory, silica has been reported to efficiently reduce surface states²⁵ that lead to a decrease in charge stability and a quenching of the fluorescence signal^{26.27}; and an additional fluorescence increase can be attributed to a larger photonic density of state in the silica shell when compared with air.²⁸

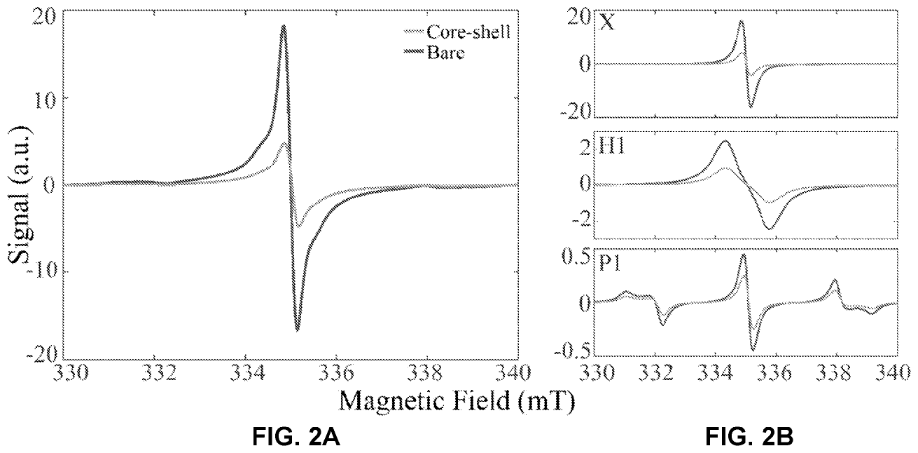

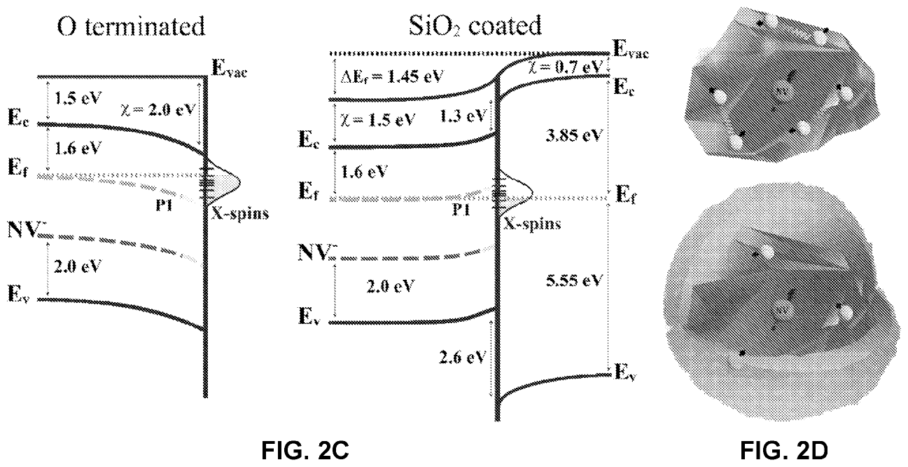

We start by investigating the type and density of paramagnetic defects present in bare and core-shell structured diamond nanocrystals. Continuous wave electron paramagnetic resonance (EPR) spectroscopy on lyophilized bare and core-shell structured nanocrystals revealed the spectroscopic signatures of at least three distinct resonances (FIG. 2A-2B). One resonance can be attributed to the presence of substitutional nitrogen defects (P1 centers) identified by their characteristic hyperfine interactions with the ¹⁴N nuclear spin.²⁹ The second resonance, henceforth referred to as X-spins, has been suggested to correspond either to dangling bonds³⁰ or negatively charged vacancies in the near-surface region³¹—for both defects, the expected g-factor lies within the precision of our spectrometer. The remaining third resonance can be assigned to a hydrogen atom-vacancy (H1) complex.³²

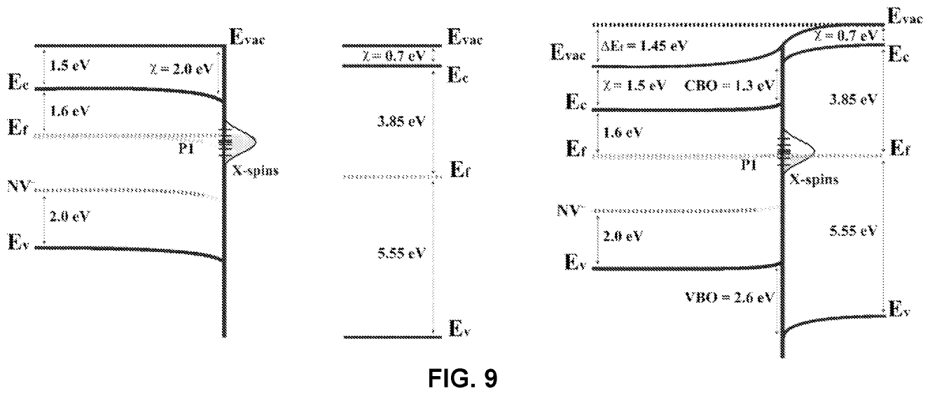

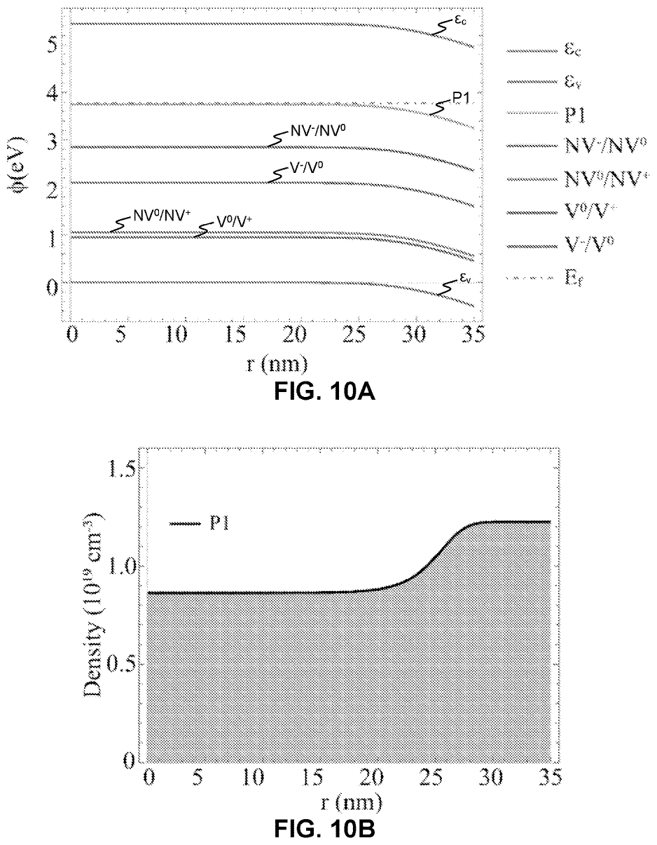

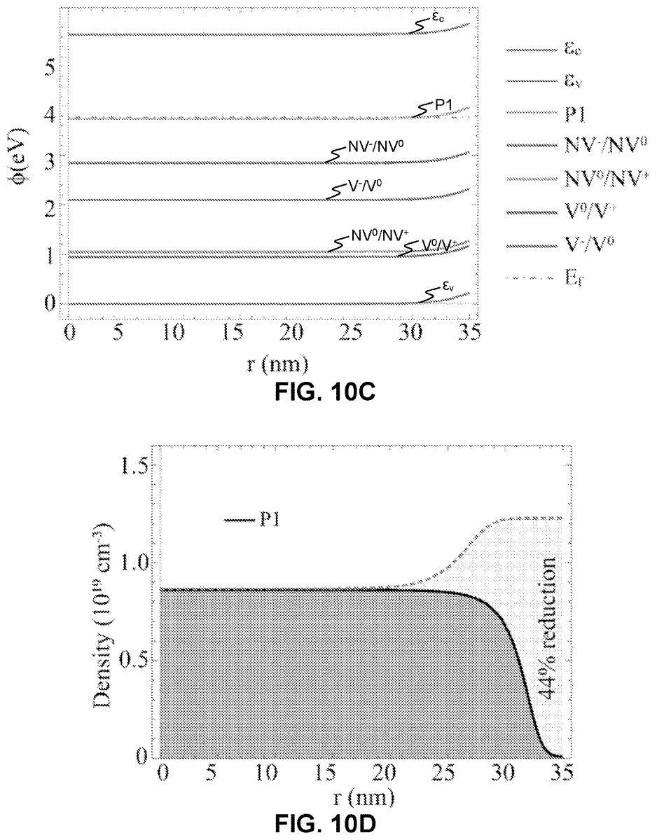

The core-shell structures showed a significant reduction of all resonances when compared to the bare diamond nanocrystals (e.g., X-spin density is reduced by 3.8×. H1 by 2.6×, and P1 by 1.8×). The depletion in X and P1 defects points toward a band-bending at the diamond-silica interface due to changes in surface potential.³³ We illustrate this effect by aligning the electronic structures of type Ib oxygen-terminated diamond (N doped at ~100 ppm) and amorphous silica (FIG. 2C and FIG. 9 for band structure and FIG. 2D for a schematic representation). The reported surface electron affinity, χₛ~2 eV, of oxygen-terminated bare diamond³⁴ leads to a downward band-bending that stabilizes P1 and X-spins (left panel). In contrast to bare diamond, silica encapsulation results in an upwards band bending, which depletes Pt and X-spins without affecting the energetically lower laying NV-charge state (right panel). In addition to the depletion of noisy paramagnetic species, the large energy barrier (~1 eV) prevents tunneling of electrons deep into the SiO₂,3⁵ which is expected to result in an increased charge stability during photoexcitation. We confirm our model with a band bending simulation based on a solution of the Poisson equation (see, e.g., FIG. 10 and other details herein). The resulting band structure suggests a 3.8 nm thick P1 depletion layer at the diamond-silica interface, which translated into a 44% decrease in the number of P1 centers per nanoparticle. As supported by our band bending model, our simulation predicts that the NV− density remains largely unaffected by our core-shell structures. We note that direct chemical conjugation of dangling bonds or displacement of paramagnetic species in the hydrolyzation layer by silica encapsulation can also result in a reduction of paramagnetic spins at the surface.³⁶

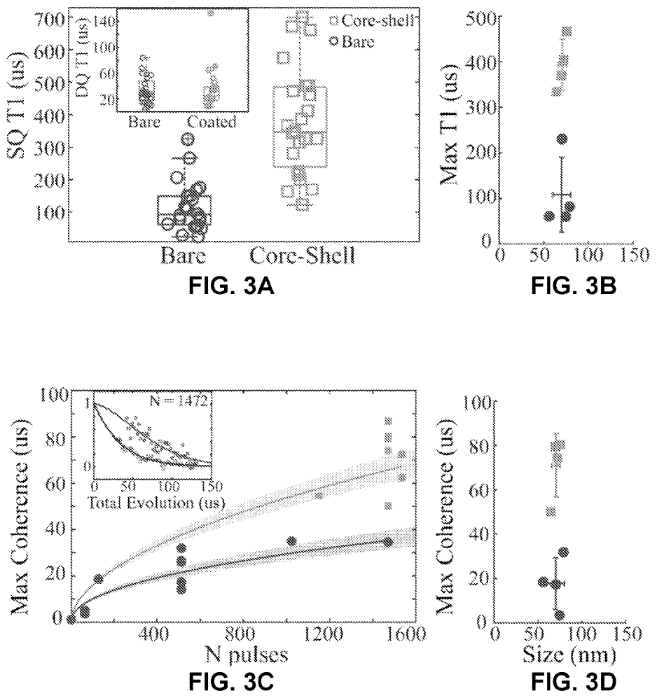

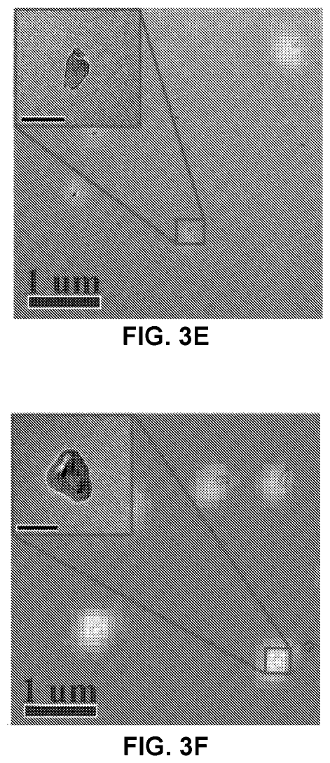

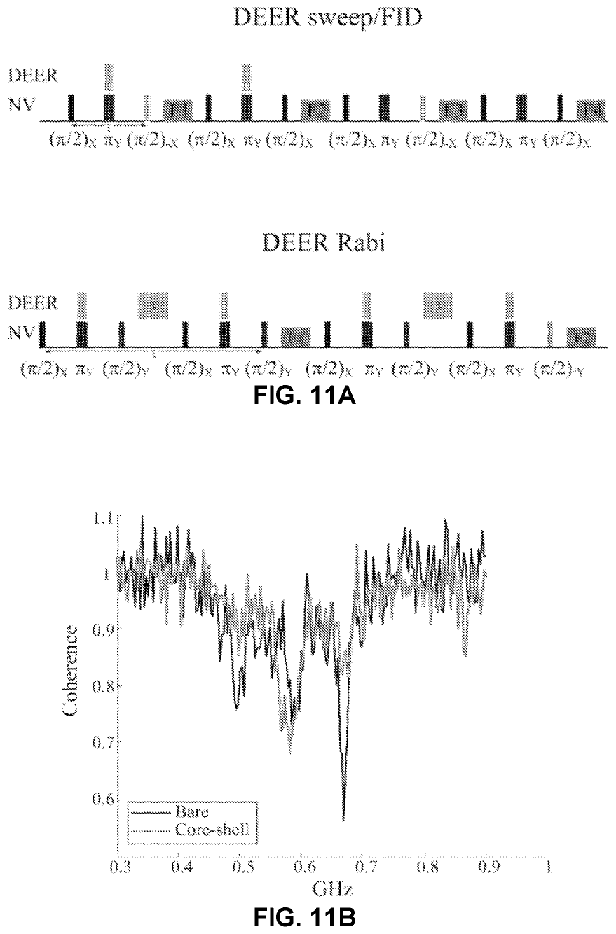

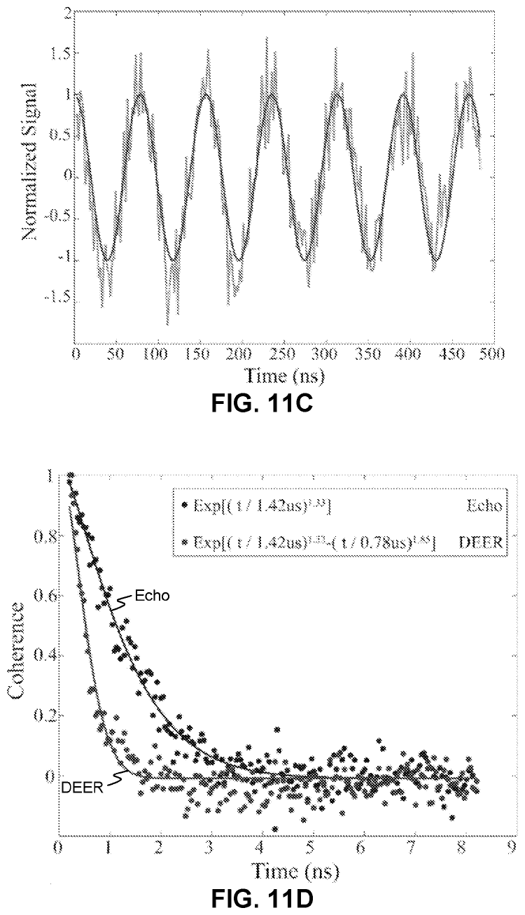

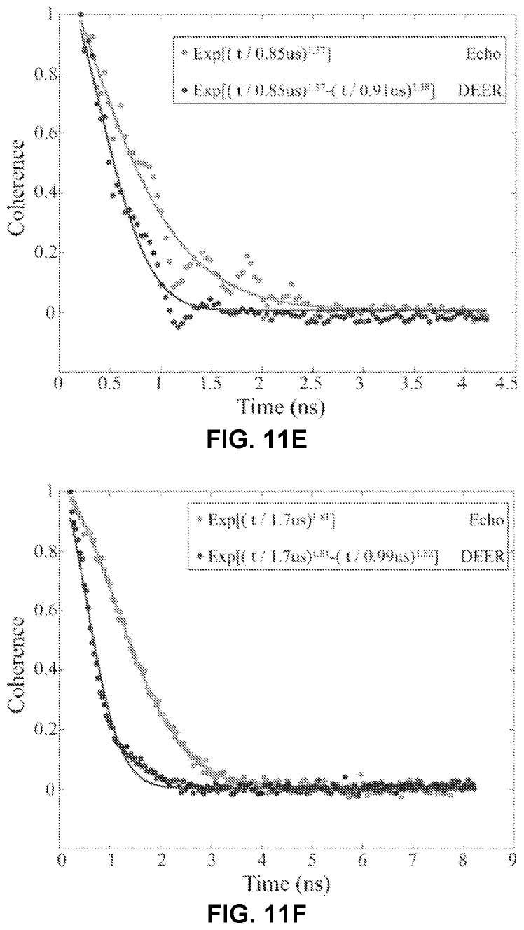

EPR spectroscopy can provide insights into the presence and density of paramagnetic defects in bare and core-shell particles. However, ensemble EPR spectroscopy does not account for particle heterogeneity and, at least in our case, does not possess the sensitivity to directly probe NV-qubits (NV centers have a ~100× reduced density compared to P1 centers²³). We overcome this challenge by selectively probing the NV spins within individual optically resolvable diamond nanocrystals. Double electron-electron resonance (DEER) measurements confirm the coupling of the NV-qubit to X-spin and P1 (FIG. 11). FIG. 3A shows the observed T₁-times for bare and core-shell diamond nanocrystals drop-casted on a glass substrate. The single quantum relaxation time (TisQ), which describes relaxations between mₛ=0 and mₛ=1, sharply increases from 114±16 μs for uncoated particles to 379±33 μs for engineered core-shell structures. However, double quantum relaxations (T₁^{DQ}), transitions between mₛ=±1, remain unaffected by coating (inset, FIG. 3A). Without wishing to be limited by mechanism ro theory, this suggests that low-frequency electronic noise is not impacted by our engineered core-shell structures. Using CLEM measurements, we confirmed that the increased NV T₁-times are not the result of selecting core-shell structures with an increased core diameter, but rather the consequence of the engineered material properties. FIG. 3C shows Tisa and core size for four bare and four core-shell particles as extracted by CLEM (FIG. 7). Although the fluorescent particles are nominally 44 nm in diameter, we observe that particles with obtainable spin coherence possess diameters of 70 nm. We point out that different characterization techniques can result in significantly different particle size estimations. For example, atomic force microscopy (AFM) measures the particle height¹⁵, whereas TEM measures the particle cross-section. In the case of disk-like particles, such as diamond nanocrystals produced by ball milling, AFM will, therefore, consistently underestimate the particle size when compared with TEM.³⁷

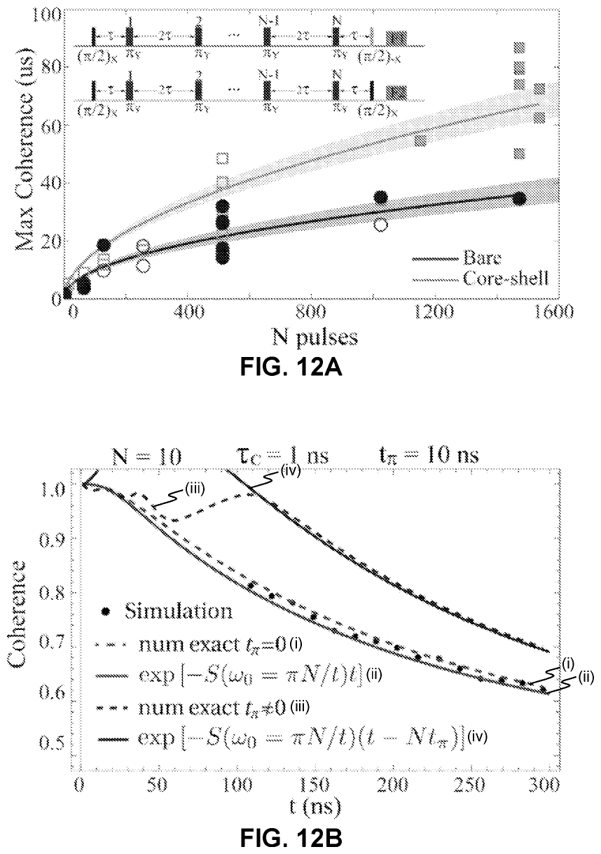

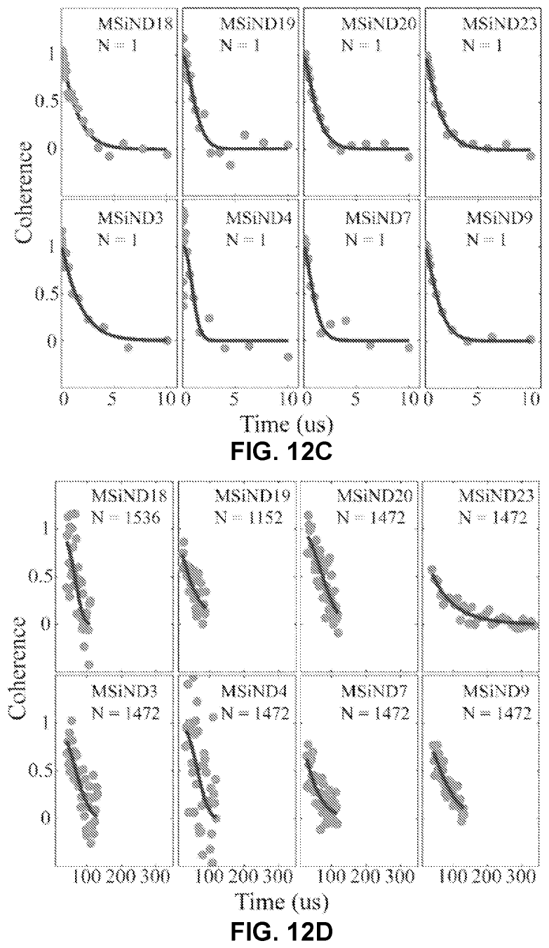

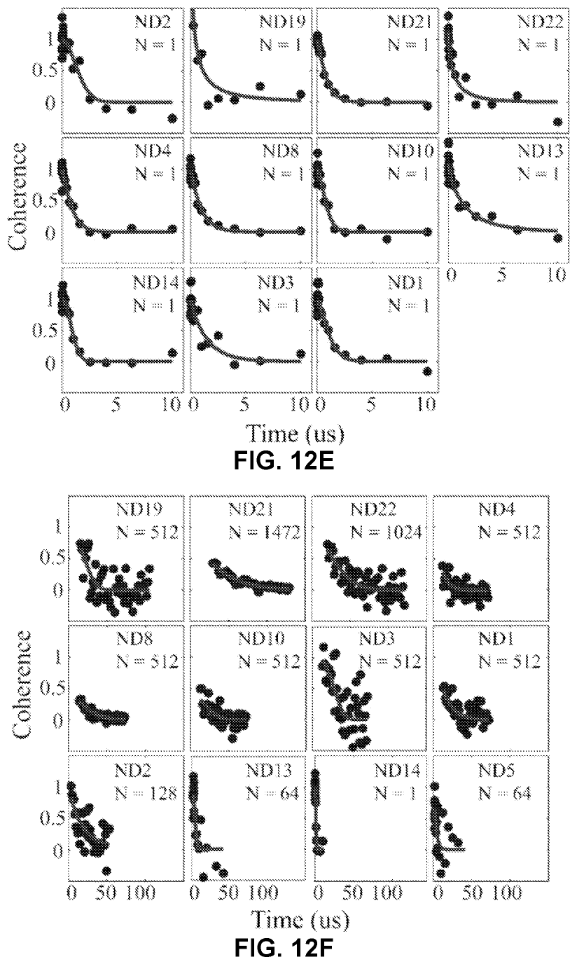

Having established that engineered core-shell structures result in an increase in qubit T₁, we next investigated its effect on T₂. From spin E_{C} ho experiments, we found that bare and coated particles have a T₂^{Echo} of 1.05±0.41 s and 1.42±0.20 s, respectively (FIG. 12C, 12E). Using Carr-Purcell-Meiboom-Gill (CPMG) dynamical decoupling,³⁸ we extend the coherence by filtering low-frequency noise with a filter function that is centered around to ω=πN/T, where N is the number of π-pulses and T is the total precession time.³⁸ We found that for bare nanocrystals, T increases with N only up to a certain level before it saturates (FIG. 12A, 12F). FIG. 3C shows the maximally achievable T₂ for twelve different bare diamond particles, with some particles showing no or marginal improvement with N (circles). For core-shell structured nanoparticles (squares), we did not observe a similar heterogeneity in T₂ and found a significant T₂ increase for all eight particles (for N>1,000, we found an average T₂^{CPMG}=70±12 μs). As a consequence, these non-limiting core-shell structures result in an average 3.5-fold increase in qubit coherence, and a 3.2-fold decrease in particle to particle T₂ variation (e.g., the relative standard deviation is

$$\frac{σ ( {T}_{2} )}{〈 {T}_{2} 〉} = 0.60$$

for bare and

$$\frac{σ ( {T}_{2} )}{〈 {T}_{2} 〉} = 0.19$$

for core-shell particles). The functional dependence of T₂ on N follows a power law, T₂(N)=T_{2,echo}Nk, with k=0.53 for core-shell and k=0 to 0.47 for bare particles. The difference in scaling suggests that the spin-bath noise in core-shell structured particles is characterized by longer correlation times compared to bare particles³⁸ (e.g., see details herein). Again, CLEM measurements (FIG. 3E-3F) of four bare and four core-shell nanoparticles confirm that the cores of the studied particles are of comparable sizes.

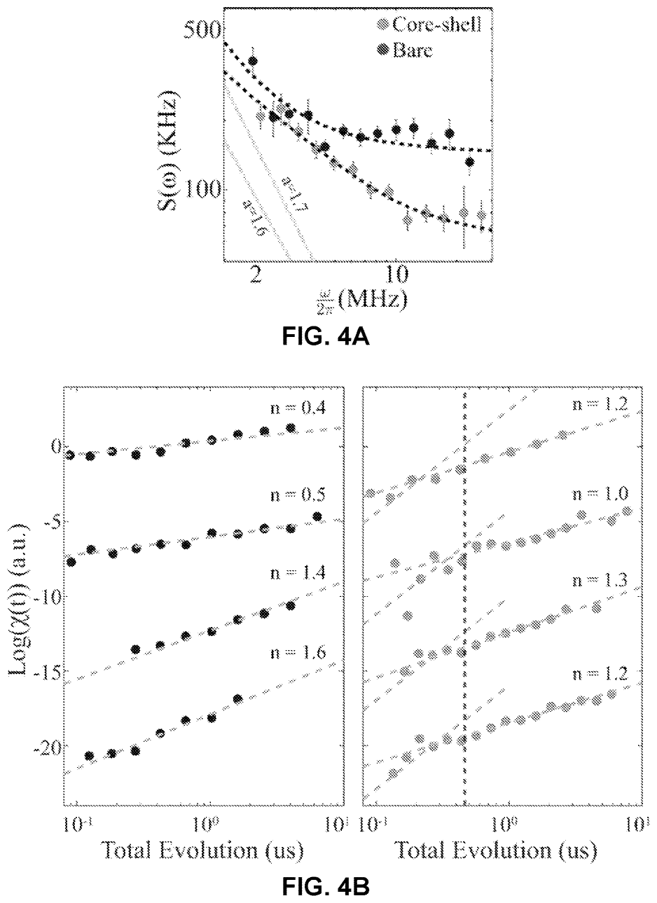

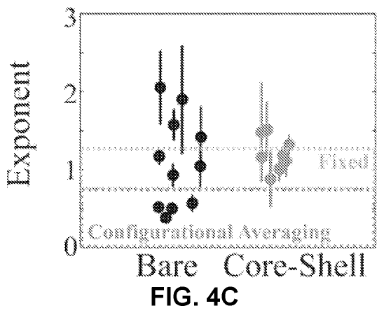

The different performance under CPMG decoupling points toward a modification of the spin noise environment in our core-shell structured particles. Assuming Gaussian noise, we reconstruct the power spectra by deconvoluting the experimentally measured CPMG time traces (see reference 38, as well as methods and details herein), which results in the solid circles in FIG. 4A. In addition to the frequency range obtained from CPMG (2 MHz to 25 MHz), we can probe high-frequency noise (~2.85 GHz) by considering the measured T₁^{SQ} spin relaxation times (circles in FIG. 4A). In the low-frequency regime (<3.7 MHz), we find that the noise power spectra of bare and core-shell structured nanocrystals are largely identical, while for higher frequencies the engineered core-shell structures show noise reduction of up to a factor 4.0×.

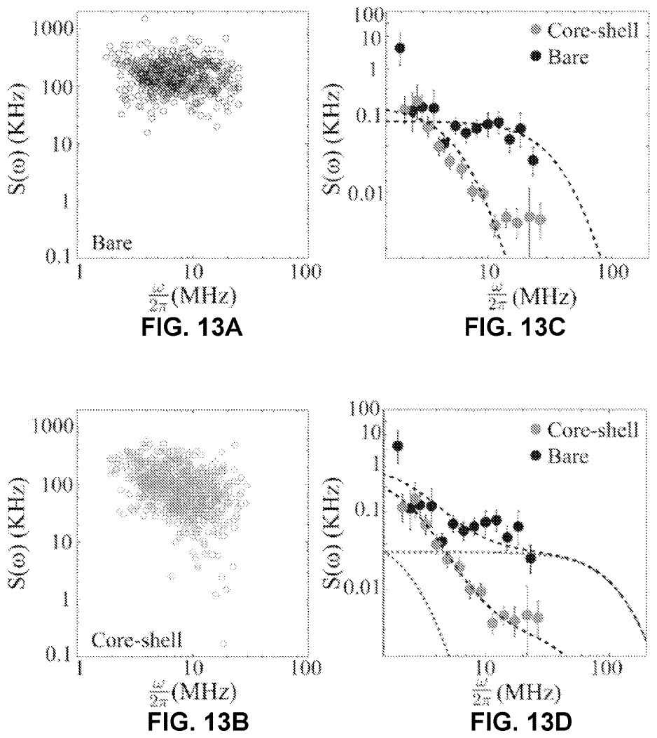

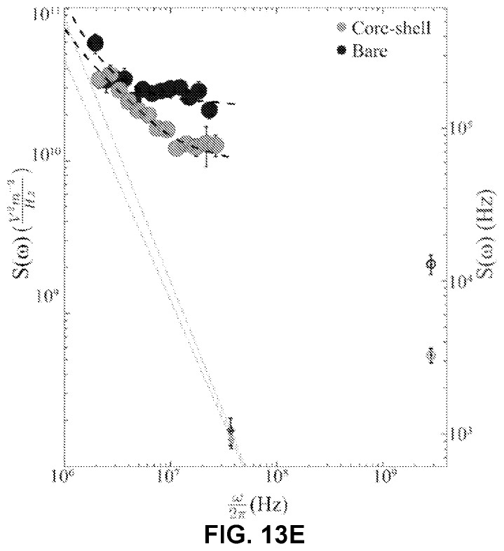

The power spectrum of a spin bath follows a Lorentzian.³⁸ For bare particles, we observed a broad power spectrum that fits a Lorentzian with short correlation times (τ_{C}≤1 ns), as would expected for a fast fluctuating surface spin bath.³⁹ In contrast, for core-shell particles this high-frequency noise is significantly reduced, revealing a Lorentzian noise spectrum with longer correlations times (τ_{C}=46±11 ns), indicating a slower evolving spin bath. At low frequencies, the power spectrum deviates from a Lorentzian noise model and instead follows a 1/f-like scaling. The exponent (a=1.7 and a=1.6 for bare and core-shell particles, respectively) of this 1/f^{a}-noise is extrapolated from the experimentally observed T₁^{DQ} and is in good agreement with results from near-surface NV centers in bulk diamond.⁴⁰ Without wishing to be limited by mechanism or theory, this suggests that charge, rather than spin, fluctuations dominate the low-frequency end of the noise power spectrum⁴ (see methods and details herein, as well as FIG. 13) and remain unaffected by our engineered core-shell structures.

Having gained an understanding of the noise spectral properties, we next turned our attention to the microscopic origin and quantum mechanical properties of the spin bath. In a Hahn E_{C} ho, the coherence factor exp(−χ(t)) follows a stretched exponential with χ(t) n, where the exponent n implicitly contains information about the spin bath.⁴¹ FIG. 4B shows four representative examples of the time evolution of χ(t) for bare and core-shell structured particles. In the case of core-shell particles, we found n~1, which is consistent with a Markovian bath where the spatial position of each bath spins remains fixed⁴¹ (FIG. 4C and additional details herein). In contrast, Hahn E_{C} ho for bare nanocrystals showed a strikingly different behavior, with n ranging from 0.4 to 2 (FIG. 4B-4C). The observation of n<1 suggests that, for bare particles, the bath spins do not remain in a fixed spatial configuration, but rather change their spatial distribution over time. A similar ‘spin-hopping’ effect is known to occur in near-surface NV centers in bulk diamond.^{42,43} Therefore, the n~1 exponent for core-shell particles suggests that the engineered shells may not only reduce the paramagnetic defects density, but may also reduce spin-hopping within the bath—an effect that has been plaguing diamond-based quantum sensing.⁴²

Pointing out the exact microscopic identity of surface-related paramagnetic defects that limit NV coherence in diamond nanocrystals and near-surface bulk systems remains an outstanding challenge. Consistent with our EPR results, recent theoretical work has proposed that surface-related sp² and sp³ dangling bonds are a major source for NV dephasing.^{25,44} We show here that engineered core-shell structures are an efficient way to suppress these surface-related defects and hence alter the spin bath properties, which leads to a significant increase in NV-qubit coherence. The presence of silica-related defects, such as hydroxyl groups, near the diamond-silica interface can furthermore lead to electron trapping⁴⁵ and so can suppress the rate of spin-hopping, an effect corroborated by modification in the stretch factor of the observed Hahn echo (FIG. 4B-4C).

We demonstrate that engineered core-shell structured particles offer an efficient means to reduce heterogeneity and extend qubit coherence. In a central spin model, the qubit coherence is inversely proportional to the spin bath density,⁴⁶ which puts the observed 3.5-fold increase in NV coherence in good agreement with the 3.88-fold reduction of X-defect density obtained by EPR spectroscopy. Combining our core-shell structures with dynamical decoupling, we extended the NV-qubit coherence to 70 μs, which approaches the coherence times of near-surface NV centers in high-purity bulk diamond.¹⁸ Furthermore, combining optical single-particle addressability with NV-based qubit sensing reveals that engineered core-shell structures reduce qubit heterogeneity and suppress complex dynamics in spatial reconfigurations of the spin bath during the course of an experiment.

The significantly decreased heterogeneity in core-shell structured particles can be directly applicable to real-world quantum sensing experiments where large variations in T₁ and T₂, and therefore sensitivity, present a challenge to experimental reproducibility. Biophysical problems ranging from nanoscale thermometry⁹ to the detection of paramagnetic species¹¹ to magnetometry¹² are expected to immediately benefit from such novel noise mitigation strategies. Depending on the measured particle, the observed increase in spin coherence and particle luminescence translates into a 4 to 120-fold reduction in signal integration time for the detection of a phase-coherent signal and a 5 to 1,000-fold reduction for the detection of an incoherent signal.⁴⁷

An effective passivation of X-spins and a desired enhancement in coherence might be achieved with thinner shells down to a few nm in thickness. Such a reduction in shell thickness will be central for sensing applications where a minimal spatial separation between the NV-qubit sensor and the target is required. Investigating different coating protocols and chemistry may further extend coherence and at the same time provide deeper insights into the microscopic nature of the X-spins. The developed techniques can directly be extended to other qubit systems, including color centers in silicon carbide,⁴⁸ lanthanide-doped nanoparticles,²¹ and quantum dots.²² Likewise, other diamond nanostructures where surface-induced NV dephasing is a limiting factor, such as in diamond-AFM tips and photonic structures, can benefit from a similar passivation approach. Finally, through standard silanization chemistry,⁴⁹ the SiO₂ surface of our core-shell particles can also serve as the basis for surface functionalization.⁴⁹ and subsequent targeting of biological molecules and structures with highly coherent qubit sensors in vivo and in vitro. Considered together, our findings emphasize the potential of engineering spin coherence using fundamental nanoscience principles to significantly improve the sensitivity of real-world nanoscale quantum sensors.

### Example 2: Non-Limiting Methods

Diamond nanocrystals: 40-45 nm diamond nanocrystals were obtained from Adimas Nanotechnologies Inc. (Raleigh, NC). In brief, type 1b microcrystals are manufactured by static high-pressure, high-temperature (HPHT) synthesis and contain about 100-200 ppm of substitutional N. These particles are milled, irradiated with 2-3 MeV electrons, and annealed at 850° C. for 2 hrs by Adamas Nanotechnologies Inc.²³

Synthesis of core-shell particles: The growth of silica shells on diamond nanocrystals was performed using a sol-gel Stober process⁵¹ from a tetraethyl orthosilicate (TEOS) precursor. To ensure uniform shell growth and prevent aggregation, we modified a polyvinylpyrrolidone (PVP) based technique.^{52,24} In particular, we improved the colloidal stability of the nanoparticles in the increasing ionic strength caused by the TEOS molecules. Briefly, 1 mg/mL of as-purchased carboxylated diamond nanocrystals (DNs) were sonicated for 20 minutes (min). In the meantime, 8 mg of PVP (10 KDa from Sigma-Aldrich, St. Louis, MO) was dissolved in 16.5 mL of reversed-osmosis purified H₂O (MQ-H₂O) and sonicated for 10 min. The nanocrystals were added into the PVP solution and stirred at −600 rpm overnight. The synthesized PVP-DNs were centrifuged at 20000×g for 30 min, and the particles were redissolved by sonicated in 3.75 mL ethanol for 20 min (at this point the solution could be stored at 4° C. until further use). The solution was stirred, and 10 μL of TEOS (Sigma-Aldrich) was added, followed by 42 μL of 30% ammonia. The solution was left to stir overnight, after which it was purified by centrifugation for 15 min at 15000×g and washed twice (10 min at 15000×g) and finally suspended with 1 mL ethanol. For long-term storage, the solution was placed in acetone instead. See, e.g., FIGS. 5-6 for illustration and chemical characterization of the synthesis process.

Characterization of particles by transmission electron microscopy (TEM): Bare and core-shell diamond nanocrystals were deposited on a copper (formvar carbon film) or silicon (Si₃N₄ film) grid while ensuring minimal aggregation (FIG. 7). Images were taken using an FEI Tecnai G2 F30 300 kV TEM.

Preparation of TEM grids: To properly address single particles, we had to prevent aggregation. The low affinity to the substrate and the high surface to volume ratio can cause the particles to agglomerate upon deposition. To increase the affinity to the substrate, the grids were treated with UV-ozone for 15 min creating a hydrophilic, negatively charged surface. The repulsion between the negative substrate and the negative particles' surface could still result in agglomeration (FIG. 7A, left panel). Therefore, for size analysis and PL measurements. 0.1 mg/mL of poly(ethyleneimine) (PEI; 2.5 KDa) in water was drop-casted on the treated grids for 2 min and wicked away. This process reversed the charge of the grids, affording much higher affinity to the particles.⁵⁴ About 0.05 mg/mL particles in water were placed on the positively charged grids for 2 min before the solution was wicked away. Grids treated this way present well dispersed particles (FIG. 7A, right panel). We noticed no effect of the PEI on the properties of the particles. To ensure no influence during coherence measurements, we avoided using PEI. Instead, after UV-ozone treatment, a drop of particles was placed on the grids that were stored in a humid container on a rocker for 30 min, after which water was wicked away. This process allowed the particles to adsorb to the surface while minimizing agglomeration due to drying.

Characterization of particles by size analysis: Particle sizes were analyzed with the help of ImageJ.⁵³ A 2-pixel Gaussian blur was applied before background subtraction. Thresholding was used to convert to a binary image from which the area (A) of an individually resolved crystal is calculated and subsequently converted to an equivalent diameter⁵⁴ (FIG. 7). Equivalent diameter. D, was extracted using D=2 \sqrt{A/π}, where A is the area of the particles in the TEM image. We analyzed core-shell particles by using a two-step thresholding process to separate the darker core from the brighter shell.

Correlated light and electron microscopy (CLEM) measurements: For CLEM measurements, nanocrystals were deposited on a silicon nitride TEM grid that was placed face-down on a glass coverslip with a fabricated coplanar waveguide. The sample was then placed in our home-built confocal microscope (see details herein) for PL and coherence measurements. Subsequent TEM images enabled us to identify individual particles by comparing particle constellations in TEM with those obtained from confocal scans. As fiducial markers for alignment of the confocal and TEM images, we used the corners and edges of the TEM grid windows, followed by overlapping of bright PL spots to the nanocrystals' TEM pattern for fine alignment (see, e.g., FIG. 7 for more details and CLEM images for PL and coherence measurements). Once we identified the particles of interest, high-magnification images were taken.

NV confocal set-up: NV measurements were performed using a home-built confocal microscope. Optical excitation was provided by a 520 nm pulsed laser (DLnsec from LABS electronics, Ludwigsburg. Germany) and focused onto the sample using a X60, NA=1.49, oil-immersion objective (Olympus APON60XOTIRF1). Translation of the excitation beam was done using a fast steering mirror (Newport FSM-300). Epifluorescence emission was separated from the excitation beam using a dichroic filter (Chroma T₆₁₀₁pxr) and filtered (Semrock LP01-594R-25) before being focused onto a single-photon counting module (Excelitas, SPCM-ARQH-14).

Photoluminescence: Photoluminescence (PL) was measured for bare and core-shell particles using our home-built confocal setup (see details herein). By drop casting a mixture of bare and core-shell particles on the same TEM grid, we were able to ensure that both particle types were measured under identical conditions (FIG. 7). Correlation with TEM then enabled us to unambiguously identify each fluorescence spot as either a bare or a core-shell particle. A total of 93 individual particles (58 core-shell and 35 bare) were analyzed. The particle radius (see size analysis in methods and SI3) in FIG. 1C was fitted to ar³, where r is the particle radius, and a is a fit parameter. We extracted a=0.65±0.11 for bare particles, and a=1.20±0.16 for core-shell particles.

Spectrum analysis: The photoluminescence (PL) spectrum of an ensemble of bare and core-shell particles with a core diameter of 70 nm was measured using Ocean Optics HR2000+ integrated into our home-built confocal microscope. We corrected for variations in the particle density by normalizing the observed PL fluorescence spectrum (PL(λ)) to one, i.e., pl(λ)=PL(λ)/Σ_{λ} PL(λ). The normalized spectrum was then fitted to pl=a pl(0)+(1−a) pl⁽⁻⁾, where a is a fit parameter with the restrictions 0≤a≤1, pl(0) is the PL spectrum of NV⁰, and pl⁽⁻⁾ is that of NV⁻.

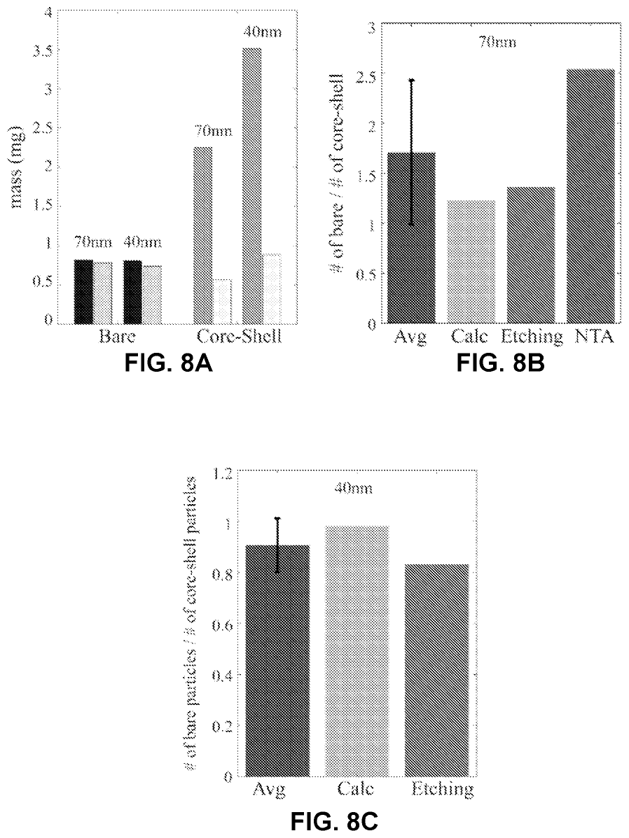

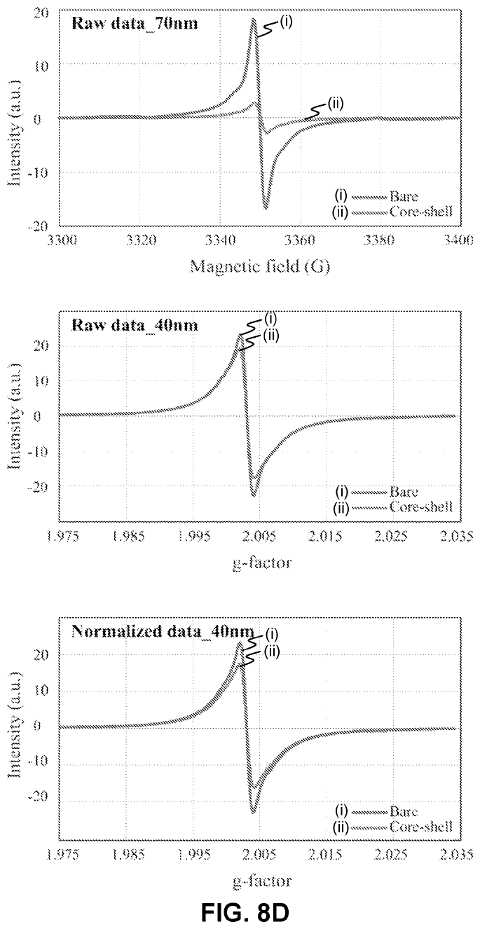

EPR measurements: EPR measurements were performed using an X-band continuous wave EPR spectrometer from Bruker (Elexsys 500) with a 100-kHz field modulation and a high-quality resonator ER 4122 SHQE. EPR spectra were collected with an incident microwave power of 2 mW, and a modulation amplitude of 0.2 mT, at room temperature. Experimental EPR spectra were decomposed into individual resonances. Fitting was done with the help of the EasySpin⁵⁵ software package which allowed us to determine spectroscopic parameters for each individual EPR signal. Measurements were done on 70 nm particles to ensure that the average particle matches the ones that were measured for coherence. Results for bare and core-shell particles were normalized both by mass and number of particles so signals for both samples could be properly compared (e.g., see FIG. 8 and additional details herein about the normalization procedure as well as results for 40 nm particles).

The measurement procedure described herein can be sensitive to the total number of particles measured in each EPR experiment. Therefore, in order to properly compare results from bare and core-shell particles, the measurements were normalized to the number of particles. For nominalization, we multiplied the EPR signal from core-shell particles by a normalization factor, F=N_{bare}/N_{CS}, where N_{bare} and N_{CS} are the number of bare and core-shell particles, respectively (FIG. 8). Since the average core diameter for the measured particles was ~70 nm (FIG. 3C), we used D_{DN}=70 nm diamond nanocrystals from Adamas for our main EPR measurements. About 1.04 mg of bare and 2.72 mg of core-shell lyophilized particles were transferred to EPR tubes for further measurements.

Analytical normalization for EPR measurements: We calculated the volume weighted density of the core-shell particles as follows: d_{CS}=(d_{DN}×V_{DN}+d_{Si}×V_{Si})/(V_{DN}+V_{Si})=2.737 g/cm³, where d_{DN}=3.3 g/cm³ and d_{Si}=2.5 g/cm³ for the densities of diamond nanocrystals and amorphous silica, respectively; and where V_{DN}=(4n/3)×(D_{DN}/2)³ and V_{Si}=Vₜₒₜₐₗ−V_{DN} are the per particle volume of the diamond core and the silica shell, respectively. We used 105 nm as the total diameter for core-shell particles). The mass and calculated density were used to extract F=N_{bare}/N_{CS}=1.23.

Nanoparticle Tracking Analysis (NTA) normalization: Due to the possible loss while transferring particles in and out of the EPR tubes, the samples were measured again, confirming ~20% loss in mass for both bare and core-shell samples. About 0.82 mg of bare and 2.25 mg of core-shell particles were dissolved in 0.82 mL and 1.25 mL of MQ-H₂O, respectively, for further analysis. About 1 μL was diluted (×10,000 for bare and X5,000 for core-shell) before 1 mL was injected to the NanosSight (Malvem Panalytical) instrument for particle count using the NTA 3.3 Dev Build 3.3.104 particle tracking software. The particles counted and the dilution factors were used to calculate F=N_{bare}/N_{CS}=2.5.

Mass etching normalization: About 250 μL of 1M KOH was added, and the solution was shaken for 12 hours (hrs) to facilitate the etching of the entire silica shell. The solution was then washed thoroughly with 3 cycles of precipitation by centrifugation (20,000 g for 30 min), removal of the supernatant, and resuspension in MQ-H₂O. The particles were then precipitated once more and were left to dry on a hot plate (90° C. for 30 mn) after supernatant removal. Finally, the mass of the dry sample was remeasured. The bare particles were processed in a similar manner to confirm negligible loss and negligible etching of the diamond core (FIG. 8A). The remaining mass (0.78 mg for bare and 0.57 mg for core-shell particles) was used to calculate F=N_{bare}/N_{CS}=1.36.

Fitting for EPR measurements: The averaged normalization factor was used to normalize the core-shell to the bare EPR signal before the data was fitted to produce g=2.0031±0.00005 (see methods described herein for fitting procedure). The data were deconvolved to extract parameters for X (g=2.0032, and linewidth, ΔB=0.3 mT), P1 (g=2.0026, ΔB=0.3mT, hyperfine coupling, A_{X}=A_{Y}=82 MHz, A_{Z}=114 MHz. and nuclear quadrupole coupling, P_{Z}=−4 MHz), and H1 (g=2.0028. ΔB=0.65 mT, and hyperfine coupling, A_{X}=A_{Y}=27.5 MHz, A_{Z}=−5.5 MHz) paramagnetic centers with S=1/2 spin. The reduction factor of area under the curve of core-shell versus bare EPR signal was calculated to be 3.81, 2.56, and 1.8 for X, P1, and H1 centers, respectively (FIG. 2B).

EPR measurements for 40 nm sample: A similar procedure was repeated with 40 nm particles (0.81 mg bare and 3.51 mg core-shell particles). Analytical (F=0.98) and mass etching (F=0.83) normalization was used to produce the data shown in FIG. 8D. P1 signal was undetectable in the 40 nm samples and instead featured a very low signature of an unknown center. The parameters for the bare sample included the following: X (g=2.0031. ΔB=0.35 mT), H1 (g=2.0031, ΔB=0.55 mT), and unknown (g=2.0032, ΔB=2 mT) paramagnetic centers. The parameters for the core-shell sample included the following: X (g=2.0031, ΔB=0.4 mT). H1 (g=2.0031, ΔB=0.4 mT), and unknown (g=2.0032, ΔB=2 mT) paramagnetic centers. The reduction factor of area under the curve of core-shell versus bare EPR signal was calculated to be 1.2, 2.6, and 0.8 for X, H1, and the unknown center, respectively. We attributed the smaller change to the probable different band structure for very small bare diamond nanocrystals, which might result in depopulation of P1 and NVs (as supported by the inability to measure P1 signal).

Electronic band structure: The band structures for diamond nanocrystals and amorphous silica were derived from literature values. We considered a type 1b (~100 ppm N impurities) oxygen-terminated diamond nanocrystal with a positive electron affinity of ~2 eV and a band gap of 5.5 eV.³⁴ The bulk conduction band, EC, lies approximately 1.5 eV below the vacuum level^{33,56} resulting in a ~0.5 eV downward band bending.⁷ The formation energy of P1 centers is positioned 1.7 eV below EC and only 0.1 eV below the Fermi level.^{33,56,58} The NV⁻ band is located 2 eV above the valence band. The band structure for amorphous silica featured a large, 9.4 eV band-gap, E_{C} that is located 0.7 eV below the vacuum level, and a fermi level that lies 5.55 eV above the valence band.^{59,60} Equalization of the fermi level (ΔE_{f}=1.45 eV) produced the band bending for alignment at the heterojunction. See, e.g., FIG. 9.

Alignment of band bending: Alignment details are provided herein. While exact values were not known for our system (type 1b HPHT carboxylated diamond nanocrystals with ~70 ppm N impurities), we assessed values from literature values for comparative systems, as described herein (e.g., for a type 1b oxygen-terminated diamond nanocrystal with ~100 ppm N impurities). The major guidelines were the ~2 eV electron affinity and downward band bending that has been previously measured in comparative systems.^{34,57} We note that all values were assessed strictly from literature and motivated but not influenced by the band bending simulation we constructed. For clarity, FIG. 9 shows the used electronic structures for our carboxylated diamond nanocrystals, the amorphous silica shell, and the heterojunction with the suggested bending effect.

Simulations of band bending: The band bending for bare and core-shell diamond nanocrystals is obtained by solving the Poisson's equation that reflects the charge density arising from electrons, holes, P1 (or ionized nitrogen), and charged states of both vacancies and NV centers. The boundary condition for the electrostatic potential is obtained by assuming charge neutrality deep in the crystal, the formation energies and densities of defects are obtained from literature,^{23,61-63} and the band bending values at the surface as extracted from the model in FIG. 2C.

Here, we present our approach to calculate the band bending of both bare and core-shell diamond nanocrystals. Band bending is caused by the spatial variation of the electrostatic potential, φ, produced by the equilibrium charge profile within the diamond.⁷⁰ Accordingly, to obtain φ, we solved the Poisson's equation ∇²_{φ}=−ρ(φ)/ϵϵ₀, where ϵ is the diamond dielectric constant, ϵ₀ is the vacuum permittivity, and ρ(φ) is the charge density arising from electrons, holes, charged states of P1, vacancies, and NV⁻ centers, namely:

$$ρ ( r , ϕ ) = - {en}_{e x} ( r , ϕ ) + e {p}_{e x} ( r , ϕ ) + e {N}_{D} ( r , ϕ ) - {eN}_{N {V}^{-}} ( r , ϕ ) + e {N}_{{NV}^{+}} ( r , ϕ ) - e {N}_{{V}^{-}} ( r , ϕ ) + e {N}_{V} + ( r , ϕ ) ,$$

where nₑₓ and pₑₓ are the electron and hole densities, respectively, N_{D} is the density of ionized nitrogen donors, N_{NV-} and N_{NV+} are the densities of the charged NV states, and N_{V-} and N_{V+} are densities of the charged vacancy states. They are given respectively by the following:

$${n}_{e x} ( r , ϕ ) = ( 2 / \sqrt{π} ) \times N c ( T ) \times {F}_{1 / 2} ( r , ϕ ) \times ( {E}_{F} - {ε}_{C} + e ϕ ( r ) ) / {k}_{B} T ,$$

$${p}_{e x} ( r , ϕ ) = ( 2 / \sqrt{π} ) \times {P}_{v} ( T ) \times {F}_{12} ( r , ϕ ) \times ( {ε}_{v} - {E}_{F} - e ϕ ( r ) ) / {k}_{B} T ,$$

$${N}_{D} ( r , ϕ ) = {( 1 - χ )}_{N} / [ exp ( [ {E}_{F} - {ε}_{N} + e ϕ ( r ) ] / {k}_{B} T ) + 1 ] ,$$

$${N}_{{NV}^{-}} ( r , ϕ ) = χ {N}_{N} / [ exp ( [ {ε}_{N {V}^{- / 0}} - {E}_{F} - e ϕ ( r ) ] / {k}_{B} T ) + 1 ] ,$$

$${N}_{{NV}^{+}} ( r , ϕ ) = χ {N}_{N} / [ exp ( [ {E}_{F} - {ε}_{N {V}^{+ / 0}} + e ϕ ( r ) ] / {k}_{B} T ) + 1 ] ,$$

$${N}_{{V}^{-}} ( r , ϕ ) = Nv / [ exp ( [ {ε}_{{V}^{- / 0}} - {E}_{F} - e ϕ ( r ) ] / {k}_{B} T ) + 1 ] , and$$

$${N}_{{V}^{+}} ( r , ϕ ) = N v / [ exp ( [ {E}_{F} - {ε}_{{V}^{+ / 0}} + e ϕ ( r ) ] / {k}_{B} T ) + 1 ] ,$$

where e>0 is the elementary charge,

$${F}_{v} ( δ ) = {\int }_{0}^{∞} {x}^{v} / [ exp ( x - δ ) + 1 ] dx$$

is the Fermi-Dirac integral, E_{F} is the Fermi level, k_{B} is the Boltzmann constant, T is the temperature, N_{N} is the density of implanted nitrogen, χ is the conversion of nitrogen to NV centers, and N_{V} is the density of vacancies. ε_{C} and εᵥ are the bottom of the conduction and top of the valance bands, respectively; and ε_{N} is the formation energy of nitrogen donors. ε_{NV}_{^{−/0}} and ε_{NV}_{^{+/0}} are the formation energy associated with the NV⁻ NV⁰ and NV⁺↔NV⁰ transitions, respectively; and ε_{V}_{^{-/0}} and ε_{V}_{^{+/0}} are the formation energy associated with V⁻↔30 V⁰ and V⁺↔V⁰ transitions, respectively. Additionally, we have N_{C}(T)=(1/4)[2m_{C}k_{B}T/(πh²)]^{3/2} and Pᵥ(T)=(1/4)[2mᵥk_{B}T/(πh²)]^{3/2}, where h is the reduced Planck's constant; and m_{C} and mᵥ are the effective electron and hole masses, respectively.

The solution for the corresponding Poisson's equation depends on the boundary conditions for φ, in addition to the Fermi level, E_{F}. The latter is obtained by assuming neutral nanocrystals deep in the interior (r≈0), where we assume φ=0. Accordingly, the value for E_{F} is obtained by imposing ρ(r=0, φ=0)=0. The other boundary condition, namely φ(r=R)=φ_{S}, defines the band bending at the diamond surface, r=R as obtained from the band alignment in FIG. 2C. Here, we assume spherical nanocrystals with radius R. For both bare and core-shell particles, we have used mₑ=0.57m₀ and mₕ=0.8m₀ with electron bare mass m₀. Other parameters included the following: E_{g}=5.45 eV as the diamond band-gap, ε_{N}=3.75 eV, ε_{NV}_{^{0/−}}=2.85 eV, ε_{NV}_{^{+/0}}=1.05 eV, ε_{V}_{^{−/0}}=2.1 eV, ε_{V}_{^{+/0}}=0.95 eV, ^{61,62}ϵ=5.8, T=300 K, N_{D}=70 ppm (15.6×10¹⁸ cm⁻³), χ=0.01, and N=20 ppm (2.8×10¹⁸ cm⁻³).^{23,63} With these values, we obtain E_{F}=3.77 eV for both bare and core-shell diamond nanocrystals. The surface electrostatic potential, φs, was set to 0.5 eV and −0.225 eV for bare and core-shell particles, respectively, as obtained from the band alignment model in FIG. 2C. In short, the upward band bending present in core-shell diamond nanocrystals (near to the surface) is responsible for shifting the P1 formation energy above the Fermi level. As a consequence, we observed a corresponding depletion of P1. Our calculation indicates a 44% reduction, which is consistent with the 45% reduction seen in our EPR measurements (FIG. 2B). The results of the simulation are shown in FIG. 10.

NV-based double electron-electron resonance (DEER) measurements: To confirm the coupling of these paramagnetic species to the NVs in our particles, we performed NV-based DEER experiments following the protocol described in reference.¹⁸ See, e.g., FIG. 11 and additional details herein. By normalizing the contrast of a DEER measurement with respect to Hahn echo decay, we removed contributions from other noise sources. Rabi frequency of the paramagnetic defects, needed for DEER free induction decay (FID), was measured using a correlation-based sequence.⁶⁴ All measurements were performed at ~205 G.

After EPR measurements demonstrated the reduction of paramagnetic species in core-shell particles, we aimed to confirm the coupling of these paramagnetic species to the NVs in our particles. The DEER protocol, described in reference¹⁸ and shown in FIG. 11, normalizes the contrast of the DEER decay with respect to Hahn echo decay to remove the effect from other noise sources. To measure the resonance frequencies of the paramagnetic defects, we kept the spacing between pulses constant (the spacing between π pulse and π/2 pulse were fixed at 200 ns) and swept the microwave frequency. To isolate the coupling from the paramagnetic defects, we modified the DEER sequence by keeping the microwave at the main paramagnetic defects resonance frequency and sweeping the duration between pulses. The normalized time trace signal (DEER decay normalized with respect to Hahn echo decay) is equivalent to free induction decay of the NV center caused by the paramagnetic defects alone.⁴² Hence, by comparing the decay rate (dephasing rate) of the normalized signal, we can compare the average paramagnetic defects coupling before and after core-shell coating. The Hahn echo decay was fitted with the following:

$${C}_{E c h o} ( τ ) = exp [ - {( τ / {T}_{2} )}^{{N}_{E}} ] .$$

The regular DEER decay part was fitted using the following:

$${C}_{D E E R} ( τ ) = exp [ - {( τ / {T}_{2} )}^{{N}_{E}} - {( τ / {T}_{FID} )}^{{N}_{FID}} ] ,$$

where T₂ and N_{E} were fitted values determined from the Hahn echo decay, and the extra decay terms T_{FID} and N_{FID} are therefore due to the paramagnetic defects alone.

DEER resonances at ~0.498 GHz, ~0.591 GHz, and ~0.669 GHz validated the coupling to P1⁷¹ (all the resonances) and X-spins (contribute to central resonance). Core-shell particles showed suppressed signal at ~0.498 GHz and ~0.669 GHz (FIG. 11B), confirming the significant reduction in P1 defects seen in EPR. We attribute the lack of reduction of the central frequency to the increased relaxation times of the surface spins that compensates for the total reduction in density. We resonantly drove the central frequency of the splittings and perform correlation measurements (lower sequence in FIG. 11A) to find a Rabi frequency for the paramagnetic defects of 12.8 MHz (FIG. 11C). DEER FID was measured to compare the contribution of paramagnetic defects to NV decoherence in bare and core-shell particles. As seen in FIG. 11D-11F, core-shell particles presented longer DEER FID times (0.91 μs and 0.99 s) compared to DEER FID times of the bare particle (0.78 s). This strongly supports the results presented in FIG. 2 and indicate a reduced density of P1 and X-spins in core-shell particles.

Coherence and relaxation measurements: All measurements were done in two different batches to ensure reproducibility. The first batch was measured using nanocrystals deposited on a #1.5 glass coverslip. The second batch was measured using nanocrystals deposited on silicon nitride TEM window grids (PELCO 15 nm or 50 nm Si₃N₄, for CLEM). Nanocrystals for measurement were chosen without favoring brighter emitters in the confocal imaging to ensure a representation of single nanocrystals with similar core sizes, as confirmed by CLEM (see, e.g., FIGS. 3E-3F and FIG. 7).

Relaxation measurements: Setting γ as the DQ transition rate and Ω as the SQ transition rate, the population dynamics of the NV triplet states can be represented by the following:

$${ρ}_{0} ( τ ) = \frac{1}{3} + ( {ρ}_{0} ( 0 ) - \frac{1}{3} ) {e}^{- 3 Ωτ} and$$

$${ρ}_{\pm 1} ( τ ) = \frac{1}{3} \pm \frac{1}{2} Δ {ρ}_{\pm 1} ( 0 ) {e}^{- ( Ω + 2 γ ) τ} - \frac{1}{2} ( {ρ}_{0} ( 0 ) - \frac{1}{3} ) {e}^{- 3 Ωτ} ,$$

where τ is the time between initialization and readout. ρₘ are the states' populations with the condition ρ₊₁=1−ρ₋₁−ρ₀, and the initialization conditions determine ρ₀(0) and Δρ_{±1}(0). We extracted

$${T}_{1}^{SQ} = \frac{1}{3 Ω} and {T}_{1}^{DQ} = \frac{1}{Ω + 2 γ} .$$

We denote the initial, i, and measured, j, time-dependent populations as ρ_{i,j}(τ) and fit the signal to the following:

$${F}_{S Q} ( τ ) = {p}_{0 , 0} ( 0 ) - {ρ}_{0 , - 1} ( 0 ) = {ae}^{- 3 Ω τ} and$$

$${F}_{D Q} ( τ ) = {ρ}_{- 1 , - 1} ( 0 ) - {ρ}_{- 1 , + 1} ( 0 ) = {ae}^{- ( Ω + 2 γ ) τ} ,$$

where a, and T₁^{SQ} or T₁^{DQ} are the fit parameters.

T₁ measurements: SQ and DQ relaxation measurements were performed using a sequence adopted from Myers et al.⁴⁰ Setting γ as the DQ transition rate and Ω as the SQ transition rate, we can extract

$${T}_{1}^{S Q} = \frac{1}{3 Ω} and {T}_{1}^{D Q} = \frac{1}{Ω + 2 γ}$$

(see additional details herein). All relaxation measurements were done at low magnetic field (e.g., ~6.7G) using 24 bare and 23 core-shell particles.

T2 measurements: Coherence measurements were performed using Hahn-Echo and CPMG sequences illustrated in FIG. 12A (inset). Randomly selected bare (n=12) and core-shell (n=8) diamond nanocrystals from two different batches (glass and silicon nitride grids) were measured for SQ and DQ relaxation times at low fields. The external magnetic field was then aligned to ~185 G for T₂ measurements starting with N=1 (echo). T₂ was measured with an increasing number of dynamical decoupling pulses up to N>1000 or saturation (see. e.g., additional details herein). The max T₂ times and corresponding N pulses are plotted in FIG. 3B. FIG. 12 shows the CPMG data collected from all the particles for T₂ echo and T₂ max.

Dynamical decoupling of T₂ measurements: Theoretical treatment of the NV spin decoherence under CPMG dynamical decoupling is given in the sections herein discussing noise spectral density. After measuring the Hahn Echo decay times, T₂ was measured with a choice of N=8, 16, 32, 64, 128, 256, 512, 1024, 1472 (or 1536) up to the point where no more improvement was seen or no contrast was detected during the set measurement time. For most particles, following Hahn Echo measurements, T₂ was measured with N=512. If contrast was detected and a decay fit was obtained, T₂ was measured for 1024≥N>1536 to obtain the maximum T₂ measured for a given particle. If a contrast was not detected, T₂ was measured for N=256, and the process repeated until an N for which contrast was detected, which was considered the saturation point. All T₂ measurements were collected ~1.5e6 times.

T₂ fitting: Coherence data, C(t), was fitted to a stretched exponential decay of the form C(t)=ae^{-χ(t)}, where χ(t)=(t/T₂)ⁿ, and a, T₂, and n are fit parameters. To account for pulse evolution time, we set the initial time, to, to be equal to the total time of T pulses, such that t₀=Nt_{π} (see FIG. 12B and additional details herein for a numerical simulation validating this protocol). To account for pulse errors, we forced the fitting parameter a to be monotonous decreasing in N. Of note, we used n as a fitting parameter and not based on a model. Fixing n=3, as would be the case of a ballistic phase evolution in a fixed spin configuration under dynamical decoupling, would significantly overestimate T₂.

Simulation for the effect of dephasing during π-pulses: It is important to emphasize that the calculations concerning T₂ decay signals, both in this work and in most other published work, consider it-pulses as delta functions. In reality, this may not be the case; and the role of the π-pulses duration on the decoherence may need to be taken into account. Biercuk et al.⁷² considered the absence of dephasing during the π-pulses. We stress that this relies exclusively on the assumption of a π-pulse length, t_{π}, that is much shorter than the correlation time of the random fluctuations, τ_{C}, i.e., t_{π}<<τ_{C}. For our system, however, we have t_{π}≈20 ns and τ_{C}≈1 ns for bare particles or τ_{C}≈50 ns for core-shell particles. For these conditions, the formulas within reference 72 cannot be employed. Accordingly, here we implemented Monte-Carlo simulations to assess completely the role of the dephasing during the application of an N number of 7-pulses. Specifically, we simulate the dephasing of a spin qubit system during the CPGM pulse sequence, under the influence of a random field

$$H / ℏ = γ {S}_{N V}^{z} {B}^{z} ( t ) .$$

We chose a random Gaussian field with correlation function [symbol] B^{z}(t))B^{z}(0) [symbol] = [symbol] δB² [symbol] e^{-t/τ}^{_{C}}, where [symbol] δB² [symbol] is a standard deviation and τ_{C} is a correlation time. We first looked at the exact (e.g., as described herein) and the approximated (C(t)≈exp[−S(ω₀)t]) decay considering the π-pulse as a delta function (t_{π}=0 ns) with N=10, τ_{C}=1 ns, and γ² [symbol] δB² [symbol] =1/400 ns. We compared these with the exact and approximated solutions for t_{π}=10 ns. We show that for our experimental conditions of t_{π}>τ_{C} and t>Nt_{π}, the formula for the coherence, C(t)≈exp[−S(ω₀)t]still holds. The results are summarized in FIG. 12B.

Noise spectral density (theory): The coherence of a qubit can be described by C(t)=exp[−χ(t)], where t is the total free precession time. Under CMPG decoupling with N π-pulses, χ(t) is given by:

$$χ ( l ) = - \frac{1}{2} {\int }_{0}^{∞} \frac{d ω}{2 π} S ( ω ) {\vert λ ( ω , t ) \vert}^{2} ,$$

where ∥λ(ω, t)|² is the filtering function.^{65,66} For N>>1, the filtering function presents a primary peak at ω=ω₀=Nπ/t, yielding C(t)≈exp[−S(ω₀)t](as further described herein). Accordingly, measuring the coherence data for different N allows us to extract the spectral noise density at different frequencies, ω₀. This method is applied for both bare and coated diamonds, with corresponding S(ω) shown in FIG. 4A. We emphasize, however, that this method assumes linear dependence of χ(t) with respect to t.

Extraction procedure for noise spectral density: To ensure the condition of N>>1 applies, we only used CPMG data with N>64. Experimentally obtained CPMG time traces (FIG. 3C inset and FIG. 12) were normalized. Values higher than 1 and lower than 0 were discarded. The cleaned data were converted to spectral density using the approximation χ(t)=tS(ω)/τ and placed on a log-log scale. The spectrum was binned into 14 logarithmic bins and plotted in FIG. 4A. See FIG. 13 for data before binning.

Placing DQ, CPMG, and SQ on the same scale for noise spectral density: The DQ and SQ relaxation measurements (CPMG dephasing and SQ relaxation) are sensitive to different noise sources, i.e., DQ relaxation is sensitive to transverse magnetic at frequencies of 18.8 MHz and parallel electric fields, whereas SQ relaxation is only sensitive to transverse 2.87 GHz. To compare QD and SQ relaxation measurements with noise spectroscopy, we followed the procedure described by Myers et al.⁴⁰

Briefly, we represented each in terms of effective E_{⊥} electric field noise spectrum, using the following:

$${S}_{{E}_{⊥}}^{S Q} ( ω ) = 2 {S}_{{E}_{‖}}^{S Q} ( ω ) = 2 \frac{{S}_{S Q} ( ω )}{{d}_{‖}^{2}} and$$

$${S}_{{E}_{⊥}}^{D Q} ( ω ) = \frac{{S}_{D Q} ( ω )}{{d}_{⊥}^{2}} ,$$

where S_{DQ}(ω)=γ(ω) and S_{SQ}(ω)=Ω(ω)) are obtained as described in the sections herein describing T₁ measurements and fitting. The procedure to obtain S_{SQ}(ω)) for CPMG data is described in the sections herein describing noise spectral density.

Fitting for noise spectral density: Using measured CPMG and DQ data, we can now fit a model for the noise spectrum. We follow a modified version of the procedure described in reference 40. The noise spectrum of a spin bath is expected to follow a Lorentzian with the generic form:

$$S ( ω ) = {\sum }_{k} ( \frac{{Δ}_{k}^{2} {τ}_{C , k}}{π ( 1 + {( ω {τ}_{c , k} )}^{2} )} ) , \qquad ( eq . 1 )$$

wherein Δₖ and τ_{C,k} are the coupling strength and bath correlation time, respectively. The power spectrum corresponding to electric noise is expected to be described by a 1/f^{a} noise.^{4,18,40,67,68}

The full fitting function for the combined noise is then given by:

$$S ( ω ) = {\sum }_{k} ( \frac{{Δ}_{k}^{2} {τ}_{C , k}}{π ( 1 + {( ω {τ}_{c , k} )}^{2} )} ) + \frac{{Δ}_{e}}{{ω}^{a}} . \qquad ( eq . 2 )$$

S(ω)=γ(ω) was obtained as discussed in the T₁ measurements and fitting section herein. The two other parameters, Δₑ and a, are related to each other, such that, Δ=S_{DQ}(ω_{DQ})*ω_{DQ}^{a}, where a is a fit parameter. The fitting process and results are illustrated in FIG. 13.

Echo exponent analysis: A detailed derivation of the functional form of the exponent of the coherence factor is described herein. The obtained stretching factor is the result of Ising interaction between the NV and a D-dimensional fluctuating spin bath.^{39,42,43,67,69} In the following, we consider two separate scenarios:

First, we consider a spin bath where the spin position remains fixed in space. The stretching factor then takes the functional dependency:

$$χ ( t ) ∝ \{ \begin{matrix}{t}^{3} & t << {τ}_{C} \\ t & t >> {τ}_{C}\end{matrix} .$$

Second, we consider a spin bath where the spin position does nor remain fixed and moved over time. Such a scenario is expected if paramagnetic centers of the bath can be ionized under laser excitation. The resulting stretching factor is then given by:

$$χ ( t ) ∝ \{ \begin{matrix}{t}^{\frac{3 D}{2 α}} & t << {τ}_{C} \\ {t}^{\frac{D}{2 α}} & t >> {τ}_{C}\end{matrix} ,$$

where D is the spatial dimensionality of the fluctuators and a is the interaction's scaling with distance (α=3 for dipole interactions and 2 for point-like charge interactions). Note, in our analysis, we consider both scenarios.

Coherence exponent analysis: The decoherence of our qubit was obtained by assuming our NV center interacting with a D-dimensional fluctuating spin bath via the Ising Hamiltonian:

$$H / ℏ = {\sum }_{j = 1}^{N} C [ {S}_{j}^{z} ( t ) {S}_{N V}^{z} / {\vert \overline{{r}_{j}} ( t ) - {\overline{r}}_{NV} \vert}^{α} ] = γ {\sum }_{j = 1}^{N} {B}_{j}^{z} ( t ) {S}_{N V}^{z} ,$$

where r̅ⱼ̅ is the position of the spin bath fluctuators, r̅_{NV} is the position of the NV-center, and C is the NV-bath interaction strength. In the presence of a dynamical decoupling sequence with equally spaced N π-pulse, the coherence of a qubit initialized as |ψP(t=0) [symbol] =(|0 [symbol] +e^{iφ}|−1 [symbol] )/\sqrt{2} is given by the following:

$$C ( t ) = 〈 exp [ i γ {\int }_{- ∞}^{∞} d τ λ ( τ ) {\sum }_{j} {B}_{j}^{z} ( t ) 〉 , \qquad ( eq . 3 )$$

where [symbol] is the ensemble average, and taking into account the effect of spin echo: λ(τ)=1 for 0≤τ≤t/2, λ(τ)=−1 for t/2≤τ≤t, and λ(τ)=0 for τ>t, τ<0. We now treat the decoherence under two different physical situations: fixed or changing spin-bath positions (r̅ⱼ̅).

Fixed position for the spins bath: For fixed bath positions, we can replace the sum of the fluctuating fields by a total fluctuating field:

$${\sum }_{j = 1}^{N} {B}_{j}^{z} = {\sum }_{j = 1}^{N} C [ {S}_{j}^{z} ( t ) / {\vert \overline{{r}_{j}} ( t ) - {\overline{r}}_{NV} \vert}^{α} ] .$$

Assuming a Gaussian character for our noise, i.e., [symbol] we can rewrite the decoherence as follows:

$$C ( t ) = exp [ - \frac{1}{2} {γ}^{2} {\int }_{- ∞}^{∞} {\int }_{- ∞}^{∞} d τ d {τ}^{′} λ ( τ ) λ ( {τ}^{′} ) 〈 {B}^{Z} ( τ ) {B}^{Z} ( {τ}^{′} ) 〉 = exp [ - χ ( t ) ] .$$

### Accordingly,

$$\begin{matrix}〈 {B}^{Z} ( τ ) {B}^{Z} ( {τ}^{′} ) 〉 = {C}^{2} {\sum }_{j , k} 〈 {S}_{j}^{Z} ( τ ) {S}_{k}^{Z} ( {τ}^{′} ) 〉 / [ {\vert \overline{{r}_{j}} - {\overline{r}}_{NV} \vert}^{α} {\vert \overline{{r}_{j}} - {\overline{r}}_{NV} \vert}^{α} ] \\ \approx {C}^{2} {\sum }_{j} 〈 {S}_{j}^{Z} ( τ ) {S}_{k}^{Z} ( {τ}^{′} ) 〉 / {\vert \overline{{r}_{j}} - {\overline{r}}_{NV} \vert}^{2 α}\end{matrix}$$

assuming the correlation persists only between same spins, i.e.,

$$〈 {S}_{j}^{Z} ( τ ) {S}_{k}^{Z} ( {τ}^{′} ) 〉 = {δ}_{j , k} 〈 {S}_{j}^{Z} ( τ ) {S}_{k}^{Z} ( {τ}^{′} ) 〉 .$$

For large number of spin-bath fluctuators, we substitute the discrete sum by an integral:

$${\sum }_{j} 1 / {\vert \overline{{r}_{j}} - {\overline{r}}_{NV} \vert}^{2 α} \to \int drn / {\vert \overline{{r}_{j}} - {\overline{r}}_{NV} \vert}^{2 α} ,$$

where n is the constant density of fluctuators.^{39,40,67,73,74} This can also be written as an integral over the frequency space, namely:

$$χ ( t ) = - \frac{1}{2} {\int }_{- ∞}^{∞} \frac{d ω}{2 π} S ( ω ) {\vert λ ( ω , t ) \vert}^{2} , with$$

$$S ( ω ) = - {γ}^{2} \int d τ {e}^{i ω τ} 〈 {B}^{Z} ( τ ) {B}^{Z} ( 0 ) 〉 ∝ \int d τ {e}^{i ω τ} 〈 {S}^{Z} ( τ ) S ( 0 ) 〉$$

$$and filtering {function}^{65.66} :$$

$${\vert λ ( ω , t ) \vert}^{2} = \{ \begin{matrix}{tan}^{2} [ ω t / ( 2 ( N + 1 ) ] [ {cos}^{2} ( ω t / 2 ) / {( ω / 2 )}^{2} ] & N : even \\ {tan}^{2} [ ω t / ( 2 ( N + 1 ) ] [ {sin}^{2} ( ω t / 2 ) / {( ω / 2 )}^{2} ] & N : odd\end{matrix} .$$

For N>>1, λ(ω, t) presents a primary peak at ω=ω₀=Nπ/t, yielding C(t)≈exp[−S(ω₀)t].

Spin Echo: For N=1 and assuming correlation function of the following form: [symbol] B^{Z}(τ)B^{Z}(τ′) [symbol] = [symbol] δB² [symbol] e^{-|τ-τ′|τ}^{_{C}} with correlation time τ_{C} and standard deviation [symbol] we obtain the following:

$$C ( t ) = exp [ \{ - {γ}^{2} 〈 δ {B}^{2} 〉 {τ}_{C} [ t - {τ}_{C} ( {e}^{- t / {τ}_{C}} - 4 {e}^{t / 2 {τ}_{C}} + 3 ) ] \} ] and$$

$$C ( t ) = \{ \begin{matrix}exp [ - {γ}^{2} 〈 δ {B}^{2} 〉 {t}^{3} / 6 {τ}_{C} ] & t << {τ}_{C} \\ exp [ - {γ}^{2} 〈 δ {B}^{2} 〉 2 t {τ}_{C} ] & t >> {τ}_{C}\end{matrix} ,$$

thus showing an early and late slope⁶⁶:

$$χ ( t ) ∝ \{ \begin{matrix}{t}^{3} & t << {τ}_{C} \\ t & t >> {τ}_{C}\end{matrix} .$$

Configurational averaging: In the derivation above, we have assumed that the position of the spin-bath fluctuators are fixed and do not change as a function of time. Although this could be accurate for some cases, reconfiguration of the spins during photoexcitation with green laser has been reported.^{42,43} To take this phenomenon into account, we employ an average over the different position configurations following references.^{42,43,69,75-77} We again start with the coherence equation (eq. 3). After assuming both Gaussian noise and

$$〈 {S}_{j}^{Z} ( τ ) {S}_{k}^{Z} ( {τ}^{′} ) 〉 = {δ}_{j , k} 〈 {S}_{j}^{Z} ( τ ) {S}_{k}^{Z} ( {τ}^{′} ) 〉 ,$$

we obtain the following:

$$C ( t ) = exp [ - \frac{1}{2} {γ}^{2} {\sum }_{j = 1}^{N} {C}^{2} / {\vert \overline{{r}_{j}} - {\overline{r}}_{N V} \vert}^{2 α} {\int }_{- ∞}^{∞} {\int }_{- ∞}^{∞} d τ d {τ}^{′} λ ( τ ) λ ( {τ}^{′} ) 〈 {S}^{Z} ( τ ) {S}^{Z} ( {τ}^{′} ) 〉 .$$

Defining F(t) as follows:

$$F ( t ) = 〈 {[ {\int }_{- ∞}^{∞} d τ λ ( τ ) {S}^{Z} ( τ ) ]}^{2} 〉 = {\int }_{- ∞}^{∞} {\int }_{- ∞}^{∞} d τ d {τ}^{′} λ ( τ ) λ ( {τ}^{′} ) 〈 {S}^{Z} ( τ ) {S}^{Z} ( {τ}^{′} ) 〉 ,$$

we obtain the following:

$$C ( t ) = {\prod }_{j = 1}^{N} exp [ - \frac{1}{2} {( γ C )}^{2} F ( t ) / {\vert \overline{{r}_{j}} - {\overline{r}}_{N V} \vert}^{2 α} ] .$$

Averaging over different positional configurations and assuming the N-spin bath fluctuators in a D-dimension volume (L^{D}), we obtain the following:

$$C ( t ) = \int … \int ( {d}^{D -} {r}_{1} / {L}^{D} ) ( {d}^{D -} {r}_{2} / {L}^{D} ) … ( {d}^{D -} {r}_{N} / {L}^{D} ) {\prod }_{j = 1}^{N} exp [ - \frac{1}{2} {( γ C )}^{2} F ( t ) / {\vert \overline{{r}_{j}} - {\overline{r}}_{N V} \vert}^{2 α} ] = \int … \int ( {d}^{D -} {r}_{1} / {L}^{D} ) exp [ - \frac{1}{2} {( γ C )}^{2} F ( t ) / {\vert {\overline{r}}_{N V} - {\overline{r}}_{1} \vert}^{2 α} ] … ( {d}^{D -} {r}_{N} / {L}^{D} ) exp [ - \frac{1}{2} {( γ C )}^{2} F ( t ) / {\vert {\overline{r}}_{N V} - {\overline{r}}_{N} \vert}^{2 α} ] = {\{ \int ( {d}^{D -} r / {L}^{D} ) exp [ - \frac{1}{2} {( γ C )}^{2} F ( t ) / \vert {\overline{r}}_{N V} - \overline{r} {\vert}^{2 α} ] \}}^{N} = {\{ 1 - \frac{1}{{L}^{D}} \int {d}^{D -} r \{ 1 - exp [ - \frac{1}{2} {( γ C )}^{2} F ( t ) / {\vert {\overline{r}}_{N V} - \overline{r} \vert}^{2 α} ] \} \}}^{N} .$$

Using now

$$exp ( - x ) = {lim}_{M \to ∞} {( 1 - x / M )}^{M} ,$$

where the fluctuator density n=N/L^{D} with L^{D}→∞ (thermodynamic limit), we obtain the following:

$$C ( t ) = exp [ - n \int {d}^{D} r \{ 1 - exp [ - \frac{1}{2} {( γ C )}^{2} F ( t ) / {\vert {r}_{N V} - r \vert}^{2 α} ] \} ] .$$

We now assume a D-hypersphere of fluctuations and r_{NV}=0, so our integral becomes as follows:

$$\int {d}^{D} r \{ 1 - exp [ - \frac{1}{2} {( γ C )}^{2} F ( t ) / {r}^{2 α} ] \} = \int d r {r}^{D - 1} \int d Ω \{ 1 - exp [ - \frac{1}{2} {( γ C )}^{2} F ( t ) / {r}^{2 α} ] \} .$$

Defining x=γC \sqrt{F(t)}/r^{α}, we get the following:

$$\frac{1}{α} \int d {Ω [ γ C \sqrt{F ( t )} ]}^{D / α} {\int }_{x 1}^{∞} d x ( 1 - exp [ - \frac{1}{2} {x}^{2} ] ) / {x}^{( D + α ) / α} ,$$

with

$$x 1 = \frac{γ C \sqrt{F ( t )}}{R} .$$

For x1→0, the integral only converges for D/α<2:

$${\int }_{x 1}^{∞} d x ( 1 - exp [ - \frac{1}{2} {x}^{2} ] ) / {x}^{( D + α ) / α} = {2}^{- ( D + 2 α ) / 2 α} Γ ( - D / 2 α )$$

and finally:

$$C ( t ) = exp [ - \{ n / ( {α2}^{( D + 2 α ) / 2 α} ) \} Γ ( - D / 2 α ) \int d {Ω ( γ C )}^{D / α} {F ( t )}^{D + 2 α} ] .$$

The time-dependent part of the exponent is given by the following:

$${F ( t )}^{D / 2 α} = {[ {\int }_{- ∞}^{∞} {\int }_{- ∞}^{∞} d τ d {τ}^{′} λ ( τ ) λ ( {τ}^{′} ) 〈 {S}^{Z} ( τ ) {S}^{Z} ( {τ}^{′} ) 〉 ]}^{D / 2 α} .$$

Repeating the analysis we did before, considering [symbol] S^{Z}(τ)S^{Z}(τ′) [symbol] = [symbol] S² [symbol] e^{-|τ-τ′|τ}^{_{C}} and N=1, we obtain: [symbol] C(t)=exp[−γ²nK×2τ_{C}{t−τ_{C}(e^{-t/τ}^{_{C}}−4e^{-t/2τ}^{_{C}}+3)}] with
K=[ [symbol] S² [symbol] /(α2^{2α+D)/2α})Γ(−D/2α)∫dΩC^{D/α}]. This results in:

$$C ( t ) = \{ \begin{matrix}exp [ - {γ}^{2} {nK ( {t}^{3} / 6 {τ}_{C} )}^{3 D / 2 α} ] & t << {τ}_{C} \\ exp [ - {γ}^{2} {nK ( 2 t {τ}_{C} )}^{3 D / 2 α} ] & t >> {τ}_{C}\end{matrix} ,$$

$$and$$

$$χ ( t ) ∝ \{ \begin{matrix}{t}^{3 D / 2 α} & t << {τ}_{C} \\ {t}^{D / 2 α} & t >> {τ}_{C}\end{matrix} .$$

As can be seen, when considering configurational averaging, the time evolution of the stretching factor has a dependence on the spatial dimensionality of the fluctuators.

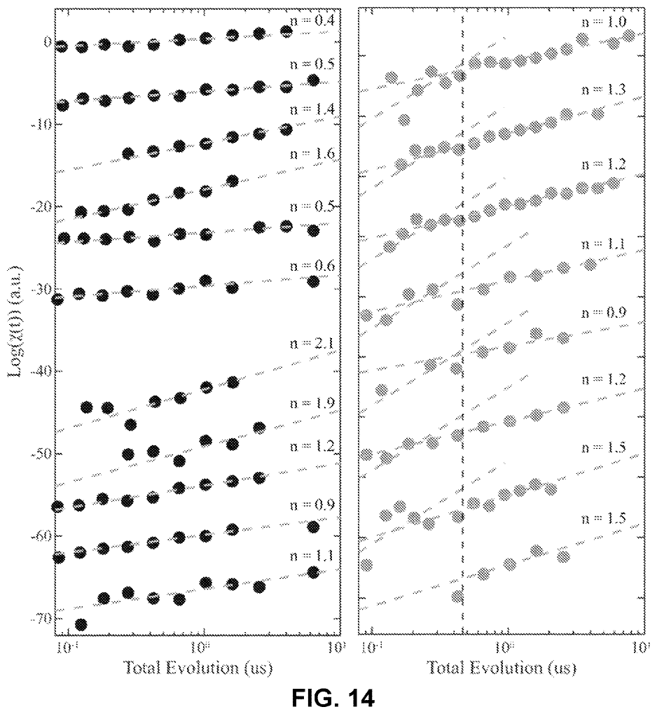

Fitting and plotting χ(t) time trace plots: A transition from the ballistic to the random walk regime occurs at τ_{C}, and leads to a double exponential decay of the echo signals. However, in a system with short correlation times, as it is the case in our diamond nanocrystals (see FIG. 4A and FIG. 13), we would expect to be primarily in the random walk regime. This prevents us from using our data for an accurate fitting in the ballistic regime. To ensure analysis within the random walk regime, we only consider evolution times larger than 10τ_{C}, which allows us to fit this truncated data to a single exponential. The bare and core-shell echo time traces were plotted (FIG. 4C and FIG. 14) using the identity Log(χ(t))=Log(−Log(C(t)). For core-shell particles in the ballistic regime we also plotted a guide to the eye with

$$χ ( t ) = {( \frac{t}{{T}_{2}} )}^{3} .$$

Statistics: Error bars represent standard error unless otherwise noted. T1 relaxation plot in FIG. 3A is illustrated using a box and whisker diagram showing the minimum, lower quarter (25th percentile), median, upper quarter (75th percentile), and maximum points of the given data. Outliers are marked with a “+” symbol and correspond to points that are more than q3+1.5(q3−q1) or less than q1−1.5(q3−q1), where q1 and q3 are the 25th and 75th percentiles of the sample data, respectively. T test for bare (n=24) and core shell (n=23) data sets for T1 (FIG. 3A) resulted in ρ=8.5E-08. T test for bare (n=11) and core shell (n=8) data sets for T2 echo (FIG. 12 and FIG. 14) resulted in ρ=0.03. T test for bare (n=12) and core shell (n=8) data sets for T2 max (FIG. 3B) resulted in ρ=4.6E-07. All analyses were done using two tails unequal variance T test.

Whilst the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references (including those listed herein), scientific articles, patent publications, and any other documents cited herein are hereby incorporated by reference for the substance of their disclosure.

### REFERENCES

1. Schirhagl, R. et al., Nitrogen-vacancy centers in diamond: nanoscale sensors for physics and biology, Annual Review of Physical Chemistry, 65:83-105 (2014).
2. McGuinness, L. P. et al., Quantum measurement and orientation tracking of fluorescent nanodiamonds inside living cells, Nat. Nanotechnol., 6:358-363 (2011).
3. Mohan, N. et al., In vivo imaging and toxicity assessments of fluorescent nanodiamonds in Caenorhabditis elegans, Nano Lett., 10:3692-3699 (2010).
4. Gardill. A. et al., Fast relaxation on qutrit transitions of nitrogen-vacancy centers in nanodiamonds, Phys. Rev. Applied, 13:034010 (2020).
5. Maze, J. R. et al., Nanoscale magnetic sensing with an individual electronic spin in diamond, Nature, 455:644-647 (2008).
6. Balasubramanian, G. et al., Nanoscale imaging magnetometry with diamond spins under ambient conditions, Nature. 455:648-651 (2008).
7. Dolde, F. et al., Electric-field sensing using single diamond spins, Nat. Phys., 7:459-463 (2011).
8. Kucsko, G. et al., Nanometre-scale thermometry in a living cell, Nature, 500:54-58 (2013).
9. Choi, J. et al., Probing and manipulating embryogenesis via nanoscale thermometry and temperature control, Proc. Natl. Acad. Sci. U.S.A, 117:14636-14641 (2020).
10. Ermakova, Y. G. et al., Thermogenetic neurostimulation with single-cell resolution, Nat. Commun., 8:15362 (2017).
11. Nie, L. et al., Quantum monitoring of cellular metabolic activities in single mitochondria, Sci Adv., 7:eabf0573 (2021).
12. Sage, D. L. et al., Optical magnetic imaging of living cells, Nature, 496:486-489 (2013).
13. Miller, B. S. et al., Spin-enhanced nanodiamond biosensing for ultrasensitive diagnostics, Nature, 587: 588-593 (2020).
14. Tisler, J. et al., Fluorescence and spin properties of defects in single digit nanodiamonds. ACS Nano, 3:1959-1965 (2009).
15. Knowles, H. S. et al., Observing bulk diamond spin coherence in high-purity Nanodiamonds, Nat. Mater., 13:21-25 (2014).
16. Wood, B. D. et al., Long spin coherence times of nitrogen vacancy centers in milled nanodiamonds. Phys. Rev. B Condens. Matter, 105:205401 (2022).
17. Andrich, P. et al., Engineered micro- and nanoscale diamonds as mobile probes for high-resolution sensing in fluid, Nano Lett., 14:4959-4964 (2014).
18. Sangtawesin, S. et al., Origins of diamond surface noise probed by correlating single-spin measurements with surface spectroscopy. Phys. Rev. X, 9:031052 (2019).
19. Ninnal. M. et al., Fluorescence intermittency in single cadmium selenide nanocrystals, Nature, 383:802-804 (1996).
20. Gargas, D. J. et al., Engineering bright sub-10-nm upconverting nanocrystals for single-molecule imaging, Nat. Nanotechnol., 9:300-305 (2014).
21. Ostrowski, A. D. et al., Controlled synthesis and single-particle imaging of bright, sub-10 nm lanthanide-doped upconverting nanocrystals, ACS Nano, 6:2686-2692 (2012).
22. Gupta, J. A. et al., Spin coherence in semiconductor quantum dots, Phys. Rev. B 20 Condens. Matter, 59:R10421-R10424 (1999).
23. Shenderova, 0. A. et al., Synthesis, properties, and applications of fluorescent diamond particles, J. Vac. Sci. Technol. B. 37:030802 (2019).
24. Rehor, I. et al., Fluorescent nanodiamonds embedded in biocompatible translucent shells, Small, 10:1106-1115 (2014).
25. Stacey, A. et al., Evidence for primal sp² defects at the diamond surface: candidates for electron trapping and noise sources, Adv. Mater. Interfaces, 6:1801449 (2019).
26. Canas, J. et al., High quality SiO₂/diamond interface in O-terminated p-type diamond MOS capacitors, Appl. Phys. Lett., 121:072101 (2022).
27. Bradac, C. et al., Effect of the nanodiamond host on a nitrogen-vacancy color-centre emission state, Snall, 9:132-139 (2013).
28. Khalid. A. et al., Lifetime reduction and enhanced emission of single photon color centers in nanodiamond via surrounding refractive index modification, Sci. Rep., 5:11179 (2015).
29. Loubser, J. H. N. & van Wyk, J. A., Electron spin resonance in the study of diamond, Rep. Prog. Phys. 41:1201 (1978).
30. Shames, A. I. et al., Defects and impurities in nanodiamonds: EPR, NMR and TEM study, J Phys. Chem. Solids, 63:1993-2001 (2002).
31. Peng, Z. et al., Investigation of near-surface defects of nanodiamonds by high-frequency EPR and DFT calculation, J. Chem. Phys., 150:134702 (2019).
32. Fionov, A. V. et al., Paramagnetic centers in detonation nanodiamonds studied by CW and pulse EPR, Chem. Phys. Lett., 493:319-322 (2010).
33. Diederich, L. et al., Electron affinity and work function of differently oriented and doped diamond surfaces determined by photoelectron spectroscopy, Surf Sci., 418:219-239 (1998).
34. Janitz, E. et al., Diamond surface engineering for molecular sensing with nitrogen-vacancy centers, J. Mater. Chem., 10:13533-13569 (2022).
35. Cañas, J. et al., High-quality SiO₂/O-terminated diamond interface: band-gap, band-offset and interfacial chemistry, Nanomaterials (Basel), 12:4125 (2022).
36. Ryan, R. G. et al., Impact of surface functionalization on the quantum coherence of nitrogen-vacancy centers in nanodiamonds, ACS Appl. Mater. Interfaces, 10:13143-13149 (2018).
37. Song, N. W. et al., Uncertainty estimation of nanoparticle size distribution from a finite number of data obtained by microscopic analysis, Metrologia, 46:480 (2009).
38. Bar-Gill, N. et al., Suppression of spin-bath dynamics for improved coherence of multi-spin-qubit systems, Nat. Commun., 3:858 (2012).
39. Rosskopf, T. et al., Investigation of surface magnetic noise by shallow spins in diamond, Phys. Rev. Lett., 112:147602 (2014).
40. Myers, B. A. et al., Double-quantum spin-relaxation limits to coherence of near-surface nitrogen-vacancy centers, Phys. Rev. Lett., 118:197201 (2017).
41. Wang, Z.-H. & Takahashi, S., Spin decoherence and electron spin bath noise of a nitrogen-vacancy center in diamond, Phys. Rev. B Condens. Matter, 87:115122 (2013).
42. Dwyer, B. L. et al., Probing spin dynamics on diamond surfaces using a single quantum sensor, PRX Quantum, 3:040328 (2022).
43. Davis, E. J. et al., Probing many-body dynamics in a two dimensional dipolar spin ensemble, arXiv:2103.12742 [quant-ph](55 pages) (2021).
44. Chou, J.-P., et al., Ah initio study of (100) diamond surface spins, arXiv:2110.07893 [quant-ph](12 pages) (2021).
45. Chua, L.-L. et al., General observation of n-type field-effect behaviour in organic semiconductors, Nature, 434:194-199 (2005).
46. Balasubramanian, G. et al., Ultralong spin coherence time in isotopically engineered diamond, Nat. Mater., 8:383-387 (2009).
47. Degen, C. L. et al., Quantum sensing, Rev. Mod. Phys., 89:035002 (2017).
48. Radulaski, M. et al., Scalable quantum photonics with single color centers in silicon carbide, Nano Lett., 17:1782-1786 (2017).
49. Singh, P. et al., Nanosilica: recent progress in synthesis, functionalization, biocompatibility, and biomedical applications, ACS Biomater. Sci. Eng., 5:4882-4898 (2019).
50. Rondin, L. et al., Surface-induced charge state conversion of nitrogen-vacancy defects in nanodiamonds, Phys. Rev. B Condens. Matter, 82:115449 (2010).
51. Stöber, W. et al., Controlled growth of monodisperse silica spheres in the micron size range, J. Colloid Interface Sci., 26:62-69 (1968).
52. Graf, C. et al., A general method to coat colloidal particles with silica, Langmuir. 19:6693-6700 (2003).
53. Schneider, C. A. et al., NIH Image to ImageJ: 25 years of image analysis, Nat. 20 Methods, 9:671-675 (2012).
54. Rehor, I. & Cigler, P., Precise estimation of HPHT nanodiamond size distribution based on transmission electron microscopy image analysis, Diam. Relat. Mater., 46:21-24 (2014).
55. Stoll, S. & Schweiger, A, EasySpin, a comprehensive software package for spectral simulation and analysis in EPR, J Magn. Reson., 178:42-55 (2006).
56. Diederich, L. et al., Photoelectron emission from nitrogen- and boron-doped diamond (100) surfaces, Surf Sci., 417:41-52 (1998).
57. Petrikovi, V. et al., Luminescence of nanodiamond driven by atomic functionalization: towards novel detection principles, Adv. Funct. Mater., 22:812-819 (2012).
58. Farrer, R. G., On the substitutional nitrogen donor in diamond, Solid State Commun., 7:685-688 (1969).
59. Astasauskas, V. et al., Optical and electronic properties of amorphous silicon dioxide by single and double electron spectroscopy, J. Electron Spectrosc. Relat. Phenom., 241:146829 (2020).
60. Stephenson, D. A. & Binkowski. N. J, X-ray photoelectron spectroscopy of silica in theory and experiment, J. Non-Cryst. Solids, 22:399-421 (1976).
61. Broadway, D. A. et al., Spatial mapping of band bending in semiconductor devices using in situ quantum sensors, Nature Electronics, 1:502-507 (2018).
62. Deik. P. et al., Formation of NV centers in diamond: a theoretical study based on calculated transitions and migration of nitrogen and vacancy related defects, Phys. Rev. B Condens. Matter, 89:075203 (2014).
63. Nunn, N. et al., Electron irradiation-induced paramagnetic and fluorescent defects in type 1b high pressure-high temperature microcrystalline diamonds and their evolution upon annealing, J Appl. Phys., 132:075106 (2022).
64. Sushkov, A. O. et al., Magnetic resonance detection of individual proton spins using quantum reporters, Phys. Rev. Lett., 113:197601 (2014).
65. Uhrig, G. S., Keeping a quantum bit alive by optimized pi-pulse sequences, Phys. Rev. Lett., 98:100504 (2007).
66. Yang, W. et al., Quantum many-body theory for electron spin decoherence in nanoscale nuclear spin baths, Rep. Prog. Phys., 80:016001 (2017).
67. Candido, D. R. & Flatte, M. E., Interplay between charge and spin noise in the near-surface theory of decoherence and relaxation of C3, symmetry qutrit spin~1 centers, arXiv:2303.13370 [quant-ph](19 pages) (2023).
68. Candido, D. R. & Flatte, M. E., Theory of spin center sensing of diffusion, arXiv:2112.15581 [cond-mat.mes-hall](7 pages) (2021).
69. Fel'dman, E. B. & Lacelle, S., Configurational averaging of dipolar interactions in magnetically diluted spin networks, J. Chem. Phys., 104:2000-2009 (1996).
70. Ashcroft, N. W. & David Mermin, N., Solid State Physics (Holt, Rinehart and Winston, 1976).
71. Wood, J. D. A. et al., Wide-band nanoscale magnetic resonance spectroscopy using quantum relaxation of a single spin in diamond, Phys. Rev. B Condens. Matter, 94:155402 (2016).
72. Biercuk, M. J. et al., Optimized dynamical decoupling in a model quantum memory, Nature. 458:996-1000 (2009).
73. Myers, B. A. et al., Probing surface noise with depth-calibrated spins in diamond. Phys. Rev. Lett., 113:027602 (2014).
74. Meriles, C. A. et al., Imaging mesoscopic nuclear spin noise with a diamond magnetometer, J. Chem. Phys., 133:124105 (2010).
75. Klauder, J. R. & Anderson, P. W., Spectral diffusion decay in spin resonance experiments, Phys. Rev., 125:912-932 (1962).
76. Mukherjee, R. et al., Accessing Rydberg-dressed interactions using many-body Ramsey dynamics, Phys. Rev. A. 94:053422 (2016).
77. Schultzen, P. et al., Glassy quantum dynamics of disordered Ising spins, Phys. Rev. B Condens. Matter 105:L020201 (2022).

1. A core-shell particle comprising:
a core comprising one or more color centers; and
a shell disposed around the core, wherein the shell comprises a metal oxide, a metalloid oxide, a semiconductor material, or an insulator.

2. The particle of claim 0, wherein the core comprises a diamond, and wherein the one or more color centers comprise a nitrogen vacancy in the diamond.

3. The particle of claim 1, wherein the core comprises silicon carbide, germanosilicate glass, silica, or LiBaF₃.

4. The particle of claim 1, wherein the shell comprises silicon oxide, aluminum oxide, titanium oxide, or a combination of any of these.

5. The particle of claim 1, further comprising an interlayer disposed on a surface of the core.

6. The particle of claim 5, wherein the interlayer comprises poly(acrylic acid) (PAA), polyvinylpyrrolidone (PVP), polyethylene glycol (PEG), cellulose (e.g., carboxy methyl cellulose), or a combination of any of these.

7. The particle of claim 1, wherein the core comprises a nanostructure.

8. The particle of claim 7, wherein the nanostructure comprises a nanoparticle.

9. A device comprising:
one or more core-shell particles of claim 1;
a source configured to irradiate the one or more color centers; and
a detector configured to detect one or more output signals emitted from the core-shell particles upon being irradiated.

10. A method of detecting a target, the method comprising:
providing a sample to an active area of the device of claim 9;
irradiating the device to excite the one or more color centers; and
detecting one or more output signals emitted from the core-shell particles upon being irradiated.

11. A method of detecting a target, the method comprising:
providing a sample to an active area of one or more core-shell particles of claim 1;
irradiating at least one core-shell particle to excite the one or more color centers; and
detecting one or more output signals emitted from the at least one core-shell particle upon being irradiated.

12. A method of preparing a core-shell particle, the method comprising:
providing a core comprising one or more color centers; and
depositing a shell disposed around the core, wherein the shell comprises a metal oxide, a metalloid oxide, a semiconductor material, or an insulator.

13. The method of claim 12, further comprising (e.g., prior to said providing the core):
providing an interlayer on a surface of the core, wherein said depositing the shell comprises depositing the shell on a top surface of the interlayer.

14. The method of claim 13, wherein the interlayer comprises poly(acrylic acid) (PAA), polyvinylpyrrolidone (PVP), polyethylene glycol (PEG), cellulose (e.g., carboxy methyl cellulose), or a combination of any of these.

15. The method of claim 12, wherein said depositing the shell comprises reacting the top surface of the interlayer with a silanizing agent.

16. The method of claim 12, wherein the core comprises a diamond, and wherein the one or more color centers comprise a nitrogen vacancy in the diamond.

17. The method of claim 12, wherein the core comprises silicon carbide, germanosilicate glass, silica, or LiBaF₃.

18. The method of claim 12, wherein the shell comprises silicon oxide, aluminum oxide, titanium oxide, or a combination of any of these.

19. The method of claim 12, wherein the core comprises a nanostructure.

20. The method of claim 19, wherein the nanostructure comprises a nanoparticle.
